(19) United States
(12) Patent Application Publication
(10) Pub. No.: US 2010/0068805 A1
(43) Pub. Date: Mar. 18, 2010

(54) XENO-FREE CULTURE CONDITIONS FOR HUMAN EMBRYONIC STEM CELLS AND METHODS THEREOF

(75) Inventors: Satish Totey, Bangalore (IN); Kumar Uday Kulkarni, Bangalore (IN); Shobhit Saxena, Bangalore (IN)

Correspondence Address: SCHWEGMAN, LUNDBERG & WOESSNER, P.A., P.O. BOX 2938, MINNEAPOLIS, MN, 55402, US

(73) Assignee: Stempeutics Research Private Limited, Bangalore (IN)

(21) Appl. No.: 12/488,178

(22) Filed: Jun. 19, 2009

Related U.S. Application Data
(63) Continuation of application No. PCT/IN2007/000594, filed on Dec. 17, 2007.

(30) Foreign Application Priority Data
Dec. 19, 2006 (IN) 2359/CHE/2006

(51) Int. Cl. C12N 5/071; C12N 5/02

(52) U.S. Cl. 435/366; 435/405

(57) ABSTRACT

The present disclosure provides novel culture system and methods for culturing and propagating hESCs in a substantially undifferentiated state for several passages. The ability to grow such cells without differentiation has important applications for therapeutic uses of ES cells for treating human disorders using tissue transplantation and/or gene therapy techniques. In particular, the disclosure further relates to conditioned medium obtained from human germ lineage derived feeder cells (GLDF). The hESC lines are derived, cultured and propagated in substantially undifferentiated state using the conditioned media from GLDF cells of the disclosure. In particular, the disclosure relates to the xeno-free derivation, culture and propagation of hESCs using conditioned medium of GLDF cells obtained thereof.

### PRIORITY

This application is a continuation under 35 U.S.C. §111(a) of international application No. PCT/1N2007/000594, filed Dec. 17, 2007 and published in English as WO 2008/075378 on Jun. 26, 2008, which application claims priority from Indian application serial No. 2359/CHE/2006 filed Dec. 19, 2006, which applications and publication are herein incorporated by reference.

### FIELD OF INVENTION

The present disclosure relates to culture and propagation of stem cells in an undifferentiated state. More particularly, the disclosure relates to a method of derivation and culture of human embryonic stem cells in a xeno-free condition.

### BACKGROUND OF INVENTION

Stem cells have the ability to divide without limit and to give rise to specialized cells. They are best described in the context of normal human development. Following the rule according to which, in ontogenesis, the younger the cell, the more pluripotent it is, it has been generally believed that embryonic stem cells are the only truly pluripotent cells, whereas adult stem cells are capable of only maintaining the homeostasis of the tissue in which they belong. Embryonic stem cells are uncommitted, pluripotent cells isolated from day 5-6 embryo. Embryonic stem cells can give rise to all somatic lineages upon differentiation and give rise to a wide variety of cell types, derived from ectodermal, mesoderm, and endodermal embryonic germ layers. Embryonic stem (ES) cells have been isolated from the blastocyst, inner cell mass or gonadal ridges of mouse, rabbit, rat, pig, sheep, primate and human embryos (Evans and Kauffman, 1981; Iannaccone et al., 1994; Graves and Moreadith, 1993; Martin, 1981; Notarianni et al., 1991; Thomson, et al.,-1995; Thomson, et al., 1998; Shamblott, et al., 1998, Heins, et al 2004,). Human embryonic stem cell (hESC) lines were first isolated by Thomson et al. 1998. These cells have the potential to produce any type of cells of the body in an unlimited quantity and can be genetically altered (Brivanulou et al. 2003).

Currently practiced ES culturing methods are mainly based on the use of feeder cell layers which secrete factors needed for stem cell proliferation, while at the same time, inhibit their differentiation. Feeder cell free systems have also been used in ES cell culturing, such systems utilize matrices supplemented with serum, cytokines and growth factors as a replacement for the feeder cell layer but have limited use.

To date, the most commonly used feeder cells are mouse embryonic fibroblasts (MEF) (Thomson et al., 1998; Reubinoff et al., 2000), which are prepared from day 13.5 post-coitum embryos of pregnant mice. However, concerns arise that contaminations, such as rodent viruses or proteins introduced by MEF, may make hESCs unsuitable for therapeutic purposes.

However, this approach of using mouse feeder cells has significant downside in derivation of human embryonic stem cell lines. To realize the enormous potential benefits of human embryonic stem cell therapy, bank of cell lines should be constructed preferably without exposing the cells to animal cells or proteins. Recently, some groups demonstrated that it is possible to culture hESCs on feeder cells that originate from human source (Richards et al., 2003; Amit et al., 2003; Cheng et al., 2003; Hovattaet al., 2003; Lee et al., 2005). Human feeders support prolonged undifferentiated growth of embryonic stem cells. However, the major disadvantage of using human embryonic fibroblasts or adult fallopian tube epithelial cells as feeder cells is that both of these cell lines have a limited passage capacity of only 8-10 times, thereby limiting the ability of a prolonged ES growth period. For a prolonged culturing period, the ES cells must be grown on human feeder cells originated from several subjects which results in an increased variability in culture conditions.

The other systems use a feeder-free environment that cultures hESCs in special media supplemented with Matrigel matrix plus MEF-conditioned medium (Xu et al 2001), fibronectin plus transforming growth factor (31 and basic fibroblast growth factor (bFGF) (Amit et al., 2004), or Matrigel in combination with activator of WNT pathway (Sato et al., 2004), respectively. Moreover, the stable and long-term culture of hESCs and the maintenance of their undifferentiated state still requires feeder cells along with the additional exogenous basic fibroblast growth factor (bFGF) (Kim et al., 2005).

Nat. Biotechnol. 18: 399-404 and Science 282: 1145-7; Reubinoff B E, Pera M F, Fong C, Trounson A, Bongso A. (2000) reports the derivation of embryonic stem cell lines from human blastocysts. Further, ES cells can be cultured on MEF under serum-free conditions using serum replacement supplemented with basic fibroblast growth factor (bFGF) (Amit M, Carpenter M K, Inokuna M S, Chiu C P, Harris C P, Waknitz M A, Itskovitz-Eldor J, Thomson J A. (2000). Clonally derived human embryonic stem cell lines maintain pluripotency and proliferative potential for prolonged periods of culture. Dev. Biol. 227: 271-8). Under these conditions the cloning efficiency of ES cells is 4 times higher than under fetal bovine serum.

Human ES cells can be cultured on human foreskin feeder layer as disclosed in U.S. patent application Ser. No. 10/368,045. Further, Xu, Ren-He in US patent application 20060014279, Jan. 19, 2006 reported feeder independent extended culture of human embryonic stem cells. Amit et al US patent application 20060051862 provided a method of establishing a feeder cells-free human embryonic stem cell line capable of being maintained in an undifferentiated, pluripotent and proliferative state.

Human ES cells can be grown and maintained using human embryonic fibroblasts or adult fallopian epithelial cells. When grown on these human feeder cells the human ES cells exhibit normal karyotypes, present alkaline phosphatase activity, express embryonic cell surface markers and retain all key morphological characteristics (Richards M, Fong C Y, Chan W K, Wong P C, Bongso A. (2002). Human feeders support prolonged undifferentiated growth of human inner cell masses and embryonic stem cells. Nat. Biotechnol. 20: 933-6). However, human embryonic fibroblasts or adult fallopian tube epithelial cells as feeder cells have a limited passage capacity, thereby limiting the ability of a prolonged ES growth period. Feeder cell free systems have also been used in ES cell culturing, such systems utilize matrices supplemented with serum, cytokines and growth factors as a replacement for the feeder cell layer.

Using feeder cells either from mouse or human is always cumbersome. Making feeder not only require great deal of time but also there is batch to batch variations since the feeder do not grow more than few passages.

### SUMMARY OF THE INVENTION

The present disclosure provides novel culture system and methods for culturing and propagating hESCs in a substantially undifferentiated state for several passages without the use of feeder cells. The ability to grow such cells without differentiation has important applications for therapeutic uses of ES cells for treating human disorders using tissue transplantation and/or gene therapy techniques. In particular, the disclosure further relates to conditioned medium obtained from human germ lineage derived feeder cells (GLDF). The hESC lines are derived, cultured and propagated in substantially undifferentiated state using the conditioned media from GLDF cells of the disclosure. In particular, the disclosure relates to the xeno-free derivation, culture and propagation of hESCs using conditioned medium of GLDF cells obtained thereof. Disclosure also relates to the derivation, propagation and culture of hESCs without addition of growth factor such as basic fibroblast growth factor that is generally thought to be required for their growth.

The first aspect of the present disclosure relates to a method of generating GLDF cells, wherein the method comprises: culturing hESCs on growth medium to obtain cells of germ lineages; culturing the cells of germ layers on a GLDF medium comprising of KO-DMEM, growth factors, serum supplement, media supplements or a combination thereof to obtain fibroblast like cells; treating fibroblast like cells to generate GLDF cells.

In yet another aspect the disclosure provides GLDF cells for culture and propagation of hESC lines in pluripotent state.

In yet another aspect the disclosure provides human GLDF that support the hESC lines in a long term in vitro culture systems.

Still another aspect of the present disclosure provides a Xeno-free culture system for culturing hESCs, wherein the culture system comprises GLDF cells; culture medium supplemented with growth factors, serum supplements, media supplements or a combination thereof.

Still another aspect of the present disclosure relates to method of culturing hESCs on the xeno-free culture system comprising human GLDF cells, wherein the cells remain undifferentiated, capable of self renewal and maintain differential potential into ectoderm, mesoderm and endoderm lineage.

In a particular aspect, the present disclosure provides a conditioned media derived from GLDF cells, wherein the medium is further supplemented with suitable constituents. Further aspect of the present disclosure provides a method of culturing hESCs on the conditioned medium obtained from GLDF cells so as to support the long term in vitro cultures of hESCs in pluripotent and undifferentiated state in a feeder free condition.

### BRIEF DESCRIPTION OF DRAWINGS

FIG. 1 is photomicrographs showing (a) day 8 embryoid bodies that were cultured for the derivation of GLDF cells (b) morphology of human GLDF cells at day 1 (c) morphology of human GLDF cells at day 3 and (d) morphology of human GLDF cells at day 5

FIG. 2 shows RT-PCR results showing the expression of differentiation markers on human GLDF cells. Expression of Nestin-220 bp, NF-L-560 bp, βIII tubulin-174 bp, NCAM-757 bp, GATA2-244 bp, GATA4-187 bp, BMP2-328 bp, BMP4-339 bp, HAND1-274 bp, β-actin-353b was screened at Passage 5 (P5)-Lane 1, Passage 10 (P10)-Lane 2, Passage 15 (P15)-Lane 3, Passage 20 (P20)-Lane 4, Passage 25 (P25)-Lane 5.

FIG. 3 shows RT-PCR results showing the expression of pluripotent markers on the human GLDF cells. Expression of Oct4-573 bp, Nanog-262 bp, Sox2-448 bp, Rex1-303 bp, TDGF1-498 bp was screened at Passage 5 (P5)-Lane 1, Passage 10 (P10)— Lane 2, Passage 15 (P15)-Lane 3, Passage 20 (P20)-Lane 4, Passage 25 (P25)— Lane 5. β-actin-353 by was used as housekeeping control.

FIG. 4 shows RT-PCR results showing the expression of fibroblast markers on human GLDF cells. Expression of Vimentin and P4H13 was screened at Passage 5 (P5)-Lane 1, Passage 10 (P10)-Lane 2, Passage 15 (P15)-Lane 3, Passage 20 (P20)-Lane 4, Passage 25 (P25)-Lane 5. β-actin-353 by was used as house keeping control.

FIG. 5 shows high expression of basic FGF in GLDF cells at Passage 5 (P5)— Lane 1, Passage 10 (P10)-Lane 2, Passage 15 (P15)-Lane 3, Passage 20 (P20)-Lane 4, Passage 25 (P25)

FIG. 6 is photomicrographs showing expression of fibroblast markers using immunocytochemistry was screened at Passage 5 (P5), Passage 10 (P10), Passage 15 (P15), Passage 20 (P20) and Passage 25 (P25). Pictures (a)-(d) shows expression of Vimentin, Pictures (e)-(h) shows the expression of Nestin and Pictures (i)-(l) shows the expression of P4H β.

FIG. 7 shows expression of cell surface markers analyzed by flow cytometry at passage-5 (P5), passage-10 (P10), passage-15 (P15), passage-20 (P20) and passage-25 (P25). The markers used for expression profiling of cell surface markers were CD 50, CD 106, CD 44, CD 54, CD 31, CD 105, CD 90, CD 73, CD 34, CD 45, CD 117, and CD 135.

FIG. 8 shows photomicrograph showing morphology of human embryonic stem cells HUES-7 on GLDF cells at Passage 10 (P10) and Passage 20 (P20).

FIG. 9 shows morphology of human embryonic stem cell line HUES-9 cultured on GLDF feeder cells at Passage 10 (P10) and Passage 20 (P20).

FIG. 10 shows RT-PCR results showing expression of pluripotent markers of human embryonic stem cells HUES-7 cultured on GLDF cells. Expression of Oct 4, Nanog, Sox 2, Rex 1, TDGF 1 and TERT was checked at Passage 5 (P5)-Lane 1, Passage 10 (P10)-Lane 2, Passage 15 (P15)-Lane 3, Passage 20 (P20)-Lane 4

### DESCRIPTION OF THE INVENTION

The ability to grow human embryonic stem cells without differentiation has important applications for therapeutic uses for treating human disorders using tissue transplantation and/or gene therapy techniques. The present disclosure provides culture system and methods for culturing and propagating human embryonic stem cells in a substantially undifferentiated state for several passages. In an aspect the disclosure provides feeder cells of human origin for the culture of hESCs. The feeder cells are derived from human germ lineage cells and hence termed human Germ Lineage Derived Feeder cells (GLDF cells). The disclosure also relates to an alternative way of culturing human embryonic stem cells (hESCs), by using conditioned medium obtained from GLDF cells. The hESC lines are derived, cultured and propagated in substantially undifferentiated state using the human GLDF cells and conditioned media derived there from.

An embodiment of the present disclosure relates to a xeno-free culture system for culturing hESCs, wherein the culture system comprises human GLDF cells and culture medium supplemented with growth factors, serum supplements, media supplements or a combination thereof.

The GLDF cells of the present disclosure are fibroblast like cells derived from hESCs.

Another embodiment of the present disclosure relates to the xeno-free culture system, wherein the growth factors in GLDF medium are selected from a group consisting of about 1-20 ng/ml transforming growth factor-β-1 (TGF-β-1); about 1-20 ng/ml epidermal growth factor (EGF), about 1-20 ng/ml brain derived neurotrophic factor (BDNF), about 1-20 ng/ml platelet derived growth factor (PDGF), Insulin, selenite, transferrin, about 5-100 ng/ml. Activin-A, about 5-100 ng/ml Activin-B, about 1-20 ng/ml Acidic FGF (fibroblast growth factor), about 2-20 ng/ml human Insulin growth factor (IGF), about 10-50 ng/ml Keratenocyte growth factor (KGF), about 5-20 ng/ml stem cell factor (SCF), about 5-20 ng/ml bone morphogenic protein (BMP4), about 10-20 ng/ml hepatocyte growth factor (HGF), about 20-100 ng/ml nerve growth factor (NGF), about 1× Insulin-transferrin-selenite, Neurotropin 3 (NT3) about 50 ng/ml, Neurotrophin 4 (NT4) about 50 ng/ml, N2B27 about 50 ng/ml and a combination thereof.

In preferred embodiment the culture medium in the xeno-free culture system comprises KO-DMEM, about 20% Serum replacement, about 2 mM glutamine, about 1-2% non-essential amino acids, about 0.1 mM beta-mercaptoethanol, about 50-100 unit/ml Penicillin and about 50-100 μg/ml Streptomycin, about 1-10 ηg basic fibroblast growth factor (bFGF) or a combination thereof.

In yet another embodiment the disclosure provides a method of culturing hESCs using Xeno-free culture system, wherein the hESCs are maintained in proliferative and undifferentiated state for about 30-60 passages, preferably/atleast 35 passages. The cultured hESCs remain pluripotent and retain the capability of differentiating into cells of ectoderm, mesoderm, and endoderm lineages.

Still another embodiment of the present disclosure relates to a conditioned medium for culturing hESCs, said medium prepared by the method comprising of culturing human GLDF cells for 24 hours on a growth medium comprising KO-DMEM, 20% human serum, 2 mM L-glutamine, 2% non-essential amino acids and 0.1 mM beta-mercaptoethanol separating the medium from the human GLDF cells to obtain conditioned medium.

Yet another embodiment of the present disclosure relates to a method of culturing hESCs on the conditioned medium derived from human GLDF cells, wherein the hESCs are maintained in proliferative and undifferentiated state for at least 35 passages.

In still another embodiment of the present disclosure provides undifferentiated, pluripotent and proliferative hESCs, wherein the cells are not only free from xeno-contaminants but also from feeder cells.

Surprisingly, it was found that the human embryonic stem cells (hESCs) can be grown for prolonged period maintaining the undifferentiated and proliferative state of the hESCs without any variability if cultured on xeno-free culture system, wherein the culture system comprises human GLDF cells and culture medium supplemented with growth factors, serum supplements, media supplements or a combination thereof.

The most commonly used feeder cells are mouse embryonic fibroblasts (MEF). However there is a risk of contaminations such as rodent viruses or proteins introduced by MEF which makes the hESc unsuitable for therapeutic use. Currently practiced hESCs culturing methods are mainly based on the use of feeder cell layers which secrete factors needed for stem cell proliferation, while at the same time inhibit their differentiation. The major disadvantage in using the feeder layer cells obtained from human source such as human embryonic fibroblast or adult fallopian tube epithelial cells hESCs is the limited passage capacity of only 8-10 times, thereby limiting the prolonged growth period. The feeder free environment for culturing the hESCs in special media is reported in the prior art but long term culture and maintenance in undifferentiated condition of the hESCs still requires the feeder cells along with the additional exogenous basic fibroblast growth factor. This problem has been solved by the present invention by providing xeno-free culture system, for the prolonged growth of hESCs in undifferentiated state. The xeno-free culture system of the present invention are capable of supporting proliferation of the hESCs in undifferentiated state without any contamination for prolonged period.

Unless specifically stated, the terms used in the specification have the same meaning as used in the art. The materials, methods, and examples are illustrative only and not intended to be limiting.

Stem Cell Therapy offers an opportunity to treat many degenerative diseases caused by the premature death or malfunction of specific cell types and the body's failure to replace or restore them. The only hope of complete recovery from such diseases at present is transplant surgery, but there are not enough donors to treat all patients and even when rare donors can be found, this is limited to a few body parts and is very expensive. Stem cells could be an ultimate hope for such un-curable diseases when no other treatment is available. Stem cells could be collected, grown and stored to provide a plentiful supply of healthy replacement tissue for transplantation into any body site using much less invasive surgery than conventional transplants.

Stem cells should be derived and maintained in an undifferentiated state for their use in tissue regeneration. Also they should remain proliferative for long term in-vitro cultures. For proliferating in an undifferentiated state the hESCs require support of mouse or human cells and supplemented with growth factors which maintain cell proliferation, inhibit hESCs differentiation and preserve pluripotency. In addition, for cell replacement and tissue regeneration therapies hESCs must be cultured in a complete animal-free environment and in the presence of well-defined culturing conditions which enable a complete reproduction of hESC cultures and make them clinically eligible. Methods known and practiced in the art are mainly based on the use of feeder cell layers which secrete factors needed for stem cell proliferation, while at the same time, inhibit their differentiation. The most commonly used feeder cells are mouse embryonic fibroblasts. However, concerns arises that contamination, such as rodent viruses or proteins are introduced into hESCs and/or hESC cultures by MEF. This may make hESCs unsuitable for therapeutic purposes.

In accordance with the foregoing objects the present disclosure provides an alternate way of culturing hESCs where the role of the feeder cells is replaced by supporting the culture on an extracellular matrix, or culturing the hESCs in a conditioned medium obtained from feeder cells. In particular the disclosure provides conditioned medium derived from human GLDF cells and method of culturing hESCs without directly using germ lineage derived feeder for human embryonic stem cell derivation, propagation and culture.

The term ‘Xeno-free’ as used herein refers to cell cultures free from any contamination from animal source other than cells of human origin, wherein the contamination may comprise virus and/or proteins and/or any entity from animal cells other than cells of human origin.

The term feeder free refers to cell cultures of hESCs free from cells of feeder layer or feeder cells.

Present disclosure, relates to feeder cells from human origin, their derivation, their use in xeno-free culture system and method of culturing hESCs on said system. In addition, the disclosure relates to conditioned media derived from human GLDF cells and method of culturing hESCs on the conditioned media. In a way, the present disclosure addresses the issue of contamination (such as rodent viruses or proteins introduced by animal derived feeder) from animal viruses and also the contamination of hESCs from feeder cells on which they are cultured. This renders the hESC lines unsuitable for clinical use and cell therapy.

In accordance with the present disclosure the method for generating human GLDF cells comprises preparing a suspension of cells from an undifferentiated hESC culture to generate embryoid bodies which comprises cells of three main germ lineages i.e endoderm, ectoderm and mesoderm. Further, the embryoid bodies are directly plated onto the solid surface of the bio-coated Petri dishes.

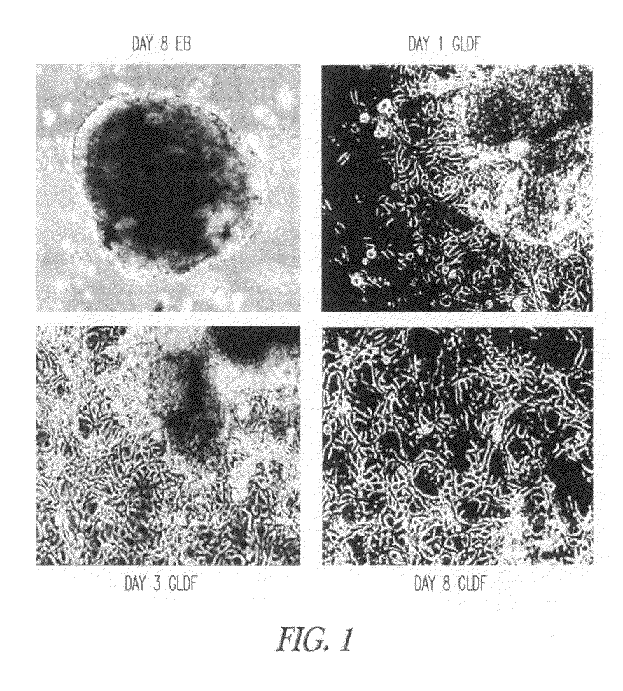

In particular aspect, the present disclosure relates to the bio-coating of the Petri dishes with about 0.1% gelatin or about 5 μg/ml collagen IV coating or about 5 μg/ml laminin coating or about 5 fibronectin coating or a combination thereof. The embryoid bodies are passaged several times on GLDF medium until they differentiate into fibroblast like cells, herein termed as human GLDF cells. FIG. 1 shows the photomicrographs of day 8 embryoid bodies that were cultured for the derivation of GLDF cells and also the morphology of human GLDF cells at day 1, day 3 and day 5. Detailed procedure of generation of human GLDF cells is provided in Example 1.

Feeder cells derived in this disclosure are derived from hESCs that differentiated into mesoderm lineages and are similar to that of mesenchymal stem cells. These cells have high telomerase activity and of embryonic origin. The feeder cells derived by this disclosure secrete all the necessary growth factors such as basic fibroblast growth factor that are required for the derivation of new hESC lines and maintaining it without any differentiation. These feeder cells can be grown and used indefinitely without any limitation of passages and have no batch to batch variations.

A preferred embodiment of the present disclosure provides human GLDF cells prepared by the method as disclosed in Example 1, wherein the GLDF cells are fibroblast like cells and are similar to cells of mesenchymal origin derived from ESC lines of human origin. Further, the GLDF cells are capable of forming a mono-layer in the cell culture. Feeder cells disclosed in the present disclosure secretes high amount of growth factors and shows high telomerase activity and hence can be used indefinitely without any limitations.

The human GLDF cells disclosed in the present disclosure are capable of supporting the growth and propagation of hESCs in a long term in vitro culture systems, wherein the stem cells are maintained in substantially undifferentiated and proliferative state.

Expression profile of human GLDF cells for various pluripotent and differentiation markers can be carried out by employing different methods known in the art.

The RT-PCR method was employed for analyzing expression profile of various differentiation markers such as Nestin, NCAM, β-III tubulin, GATA2, GATA-4, BMP2, BMP4, Hand1, Vimentin and NF light chain (See Table 1). FIGURE-2 shows the RT-PCR results showing the expression of Nestin-220 bp, NF-L-560 bp, βIII tubulin-174 bp, NCAM-757 bp, GATA2-244 bp, GATA4-187 bp, BMP2-328 bp, BMP4-339 bp, HAND1-274 bp at different passages wherein β-actin-353b was used as house keeping control. Upstream and downstream primers were used to screen the expression of various markers as below:

| | |
|---|---|
| Nestin | |
| SEQ ID: 1- | AACAGCGACGGAGGTCTCTA |
| SEQ ID: 2- | TTCTCTTGTCCCGCAGACTT |
| NCAM | |
| SEQ ID: 3- | CAGTCCGTCACCCTGGTGTGCGATGC |
| SEQ ID: 4- | CAGAGTCTGGGGTCACCTCCAGATAGC |
| β-III tubulin | |
| SEQ ID: 5- | CTTGGGGCCCTGGGCCTCCGA |
| SEQ ID: 6- | GCCTTCCTGCAGTGGTACACGGGCG |
| GATA2 | |
| SEQ ID: 7- | TGACTTCTCCTGCATGCACT |
| SEQ ID: 8- | AGCCGGCACCTGTTGTGCAA |
| GATA4 | |
| SEQ ID: 9- | TCCAAACCAGAAAACGGAAG |
| SEQ ID: 10- | CTGTGCCCGTAGTGAGATGA |
| BMP2 | |
| SEQ ID: 11- | TGTATCGCAGGCACTCAGGTCAG |
| SEQ ID: 12- | AAGTCTGGTCACGGGGAAT |
| BMP4 | |
| SEQ ID: 13- | GTCCTGCTAGGAGGCGCGAG |
| SEQ ID: 14- | GTTCTCCAGATGTTCTTCG |
| Hand1 | |
| SEQ ID: 15- | 5′-TGCCTCAGAAAGAGAACCAG |
| SEQ ID: 16- | 5′-ATGGCAGGATGAACAAACAC |
| Vimentin | |
| SEQ ID: 17- | TGCAGGACTCGGTGGACTT |
| SEQ ID: 18- | TGGACTCCTGCTTTGCCTG |
| NF light chain | |
| SEQ ID: 19- | ACGCTGAGGAATGGTTCAAG |
| SEQ ID: 20- | TAGACGCCTCAATGGTTTCC |
| β-actin | |
| SEQ ID: 21- | GCTCGTCGTCGACAACGGCT |
| SEQ ID: 22- | CAAACATGATCTGGGTCATCTTCTC |

Human GLDF cells however, do not express any pluripotent markers. The expression of various pluripotent markers was checked by performing RT-PCR. The cells were found negative for the expression of pluripotent markers such as NANOG, SOX-2, REX-1, TDGF-1 and TERT (See Table: 2). FIG. 3 shows RT-PCR results for the expression of Nanog-262 bp, Sox2-448 bp, Rex1-303 bp, TDGF1-498 bp at different passages, wherein Beta-actin marker gene was used as a house keeping control. Upstream and downstream primer sequences for expression of Beta-actin marker gene are as shown in SEQ ID NO.: 21 and SEQ ID NO.: 22. The primer sequences used to screen the expression of various markers are as below:

| | |
|---|---|
| NANOG | |
| SEQ ID: -23 | CCTCCTCCATGGATCTGCTTATTCA |
| SEQ ID: -24 | CAGGTCTTCACCTGTTTGTAGCTGAG |
| SOX-2 | |
| SEQ ID: -25 | CCCCCGGCGGCAATAGCA |
| SEQ ID: -26 | TCGGCGCCGGGGAGATACAT |
| REX-1 | |
| SEQ ID: -27 | GCGTACGCAAATTAAAGTCCAGA |
| SEQ ID: -28 | CAGCATCCTAAACAGCTCGCAGAAT |
| TDGF-1 | |
| SEQ ID: -29 | GCCCGCTTCTCTTACAGTGTGATT |
| SEQ ID: -30 | AGTACGTGCAGACGGTGGTAGTTCT |
| TERT | |
| SEQ ID: -31 | AGCTATGCCCGGACCTCCAT |
| SEQ ID: -32 | GCCTGCAGCAGGAGGATCTT |

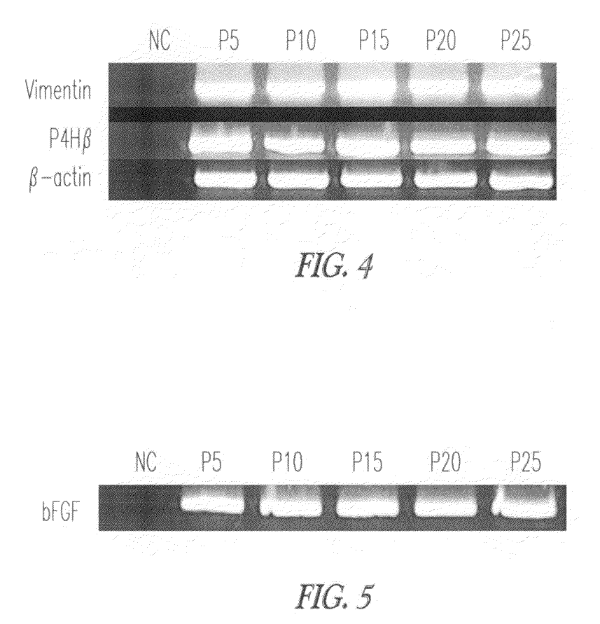

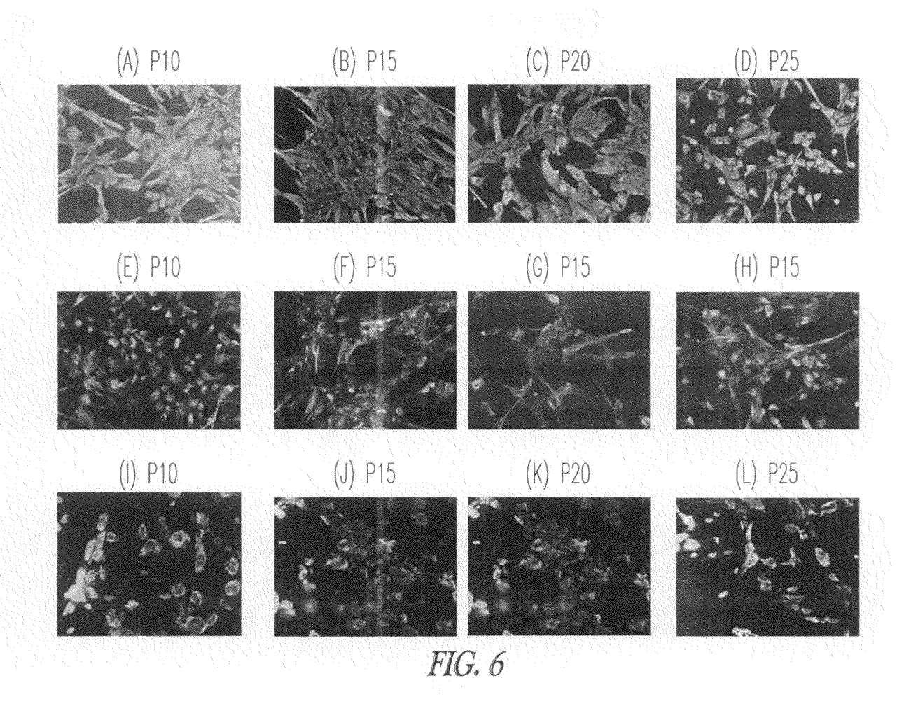

The human GLDF cells disclosed are also found to be positive for the expression of fibroblastic phenotypes as checked by RT-PCR (See Table 3). FIG. 4 shows RT-PCR results for the expression of P4Hβ and the main intermediate filament protein-Vimentin at different passages. β-actin-353 by was used as house keeping control, the expression of which was brought about by using primer sequences as shown in SEQ ID NO.: 21 and SEQ ID NO.: 22. Primer sequences for Vimentin are as shown in SEQ ID NO.: 17 and SEQ ID NO.: 18. Further the upstream and downstream primer sequences for P4Hβ are as shown below:

| | |
|---|---|
| P4Hβ | |
| SEQ ID NO.: 33- | GACAAGCAGCCTGTCAAGG |
| SEQ ID NO.: 34- | ACCATCCAGCGTGCGTTCC |

The expression of basic Fibroblast Growth Factor (bFGF) by human GLDF cells was separately checked at passage 5, 10, 15, 20 and 25 employing RT-PCR (See FIG. 5), wherein the primer sequences used are as shown below:

| | |
|---|---|
| bFGF | |
| SEQ ID NO.: 35- | GCCACATCTAATCTCATTTCACA |
| SEQ ID NO.: 36- | CTGGGTAACAGCAGATGCAA |

The expression of various fibroblast markers on human GLDF cells was also found positive as checked by immunocytochemistry. FIG. 6 shows photomicrographs for the expression of Vimentin, Nestin and P4H β. Immunocytochemistry was carried out after different passages in order to study the up-regulation and down-regulation of the genes.

The expression profiling of differentiation markers was again performed using RT-PCR. The expression of markers specific for Ectoderm cell lineages, Endoderm cell lineages and Mesoderm cell lineages was checked and were found positive for lineage specific markers.

In accordance with the present disclosure the human GLDF cells are further characterized for Ectoderm markers and were found positive for markers selected from the group consisting of NCAM and beta III tubulin, nestin, MAP2.

In accordance with the present disclosure the human GLDF cells are characterized for Endoderm markers and were found positive for markers selected from the group consisting of GATA2, FLk1, alpha actinin.

In accordance with the present disclosure the human GLDF cells are characterized for mesoderm markers and were found positive for markers selected from the group consisting of Hand1, BMP4, Brachyury, Hnf4, Hnf beta, Foxa2

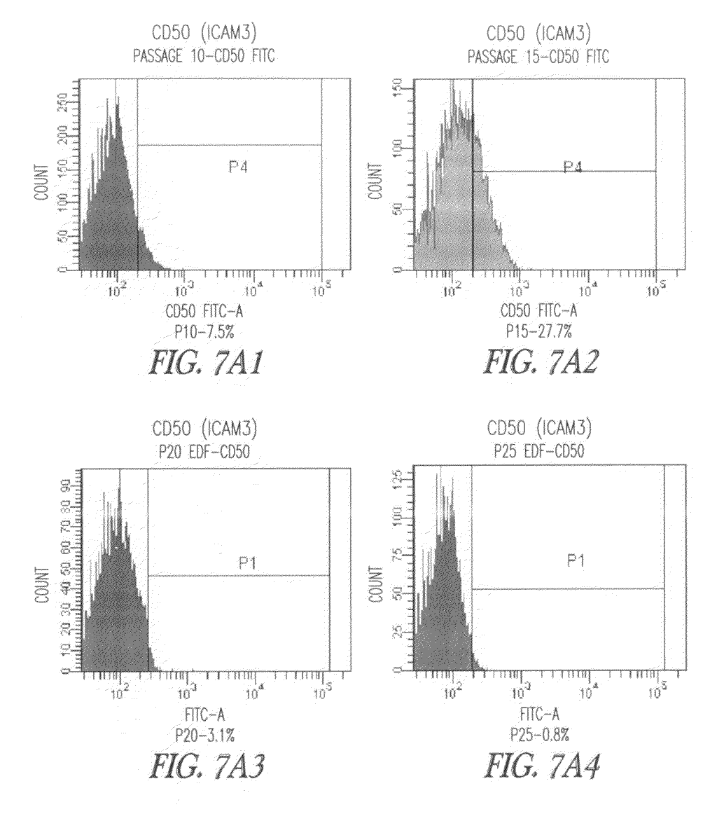

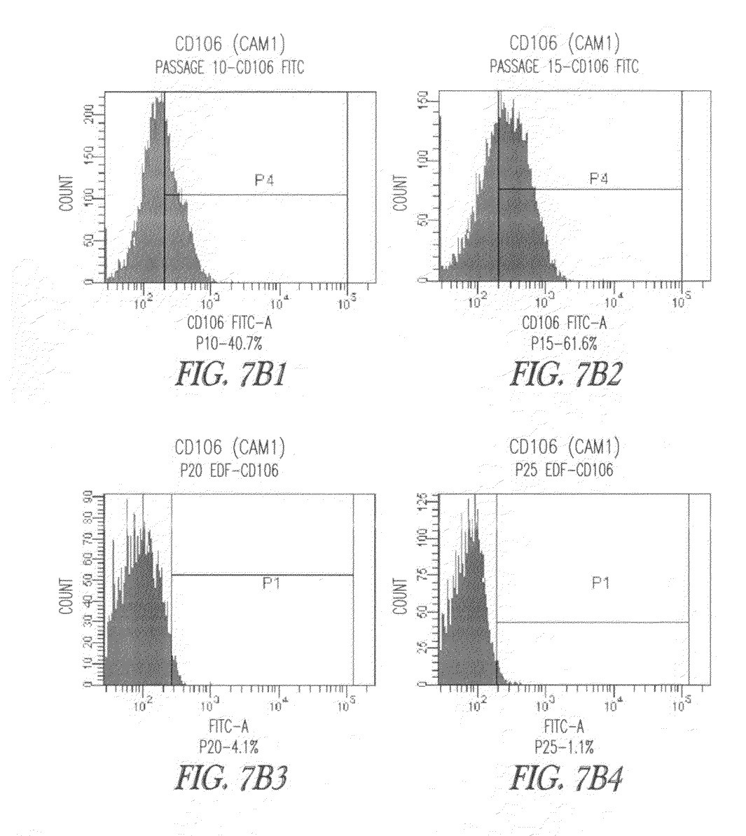

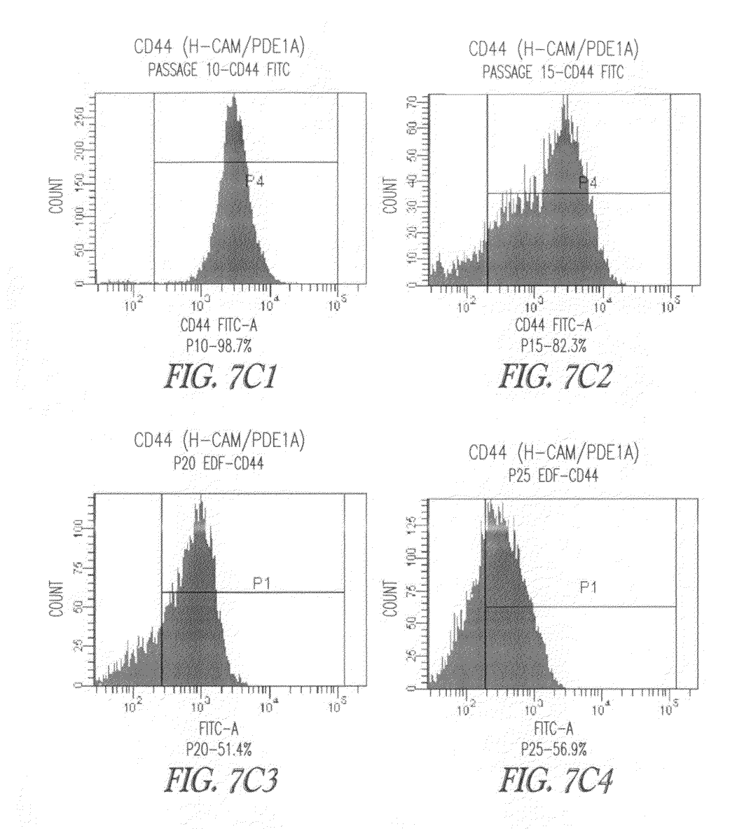

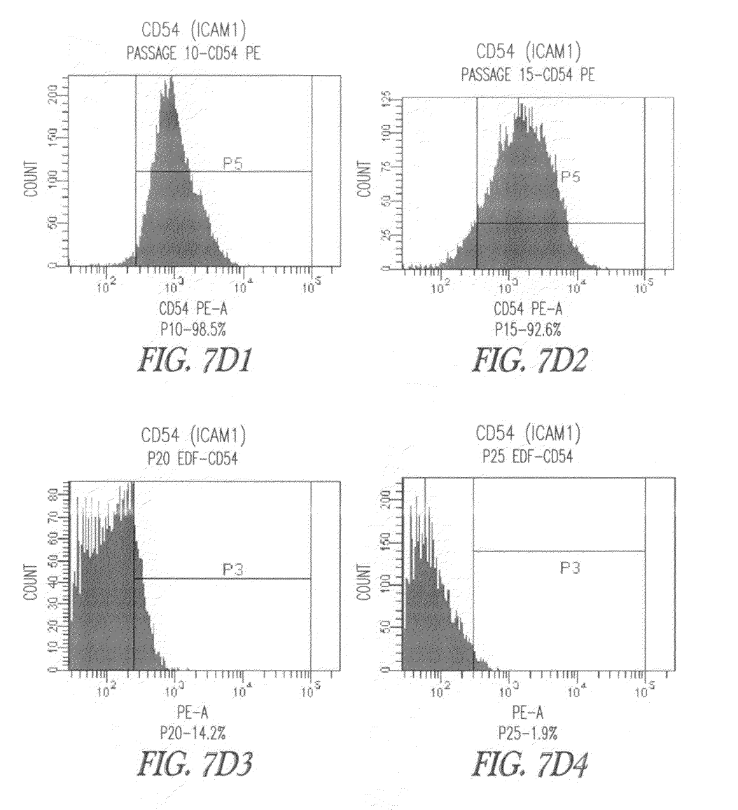

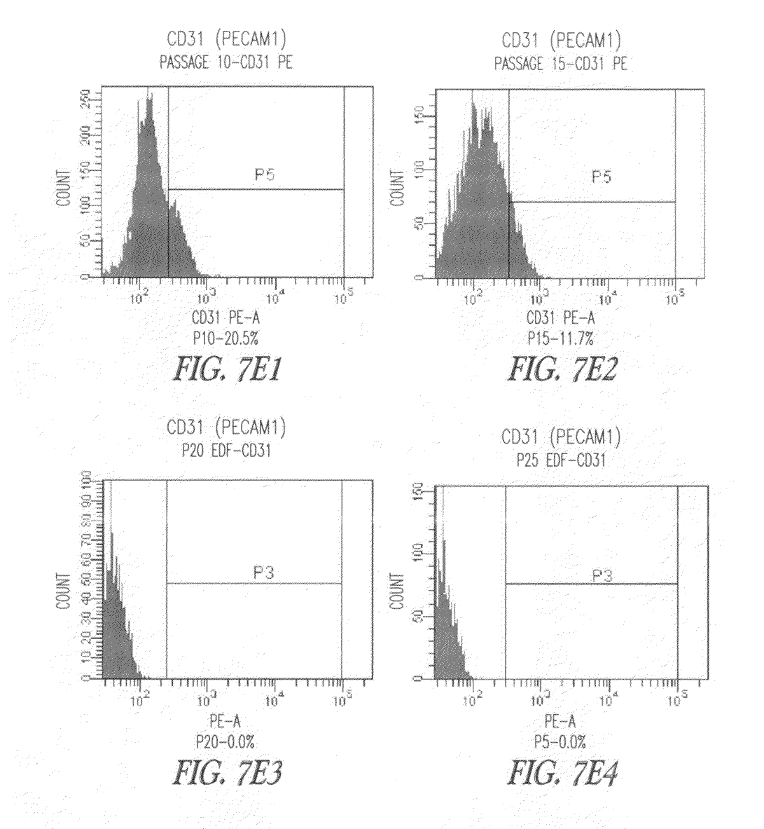

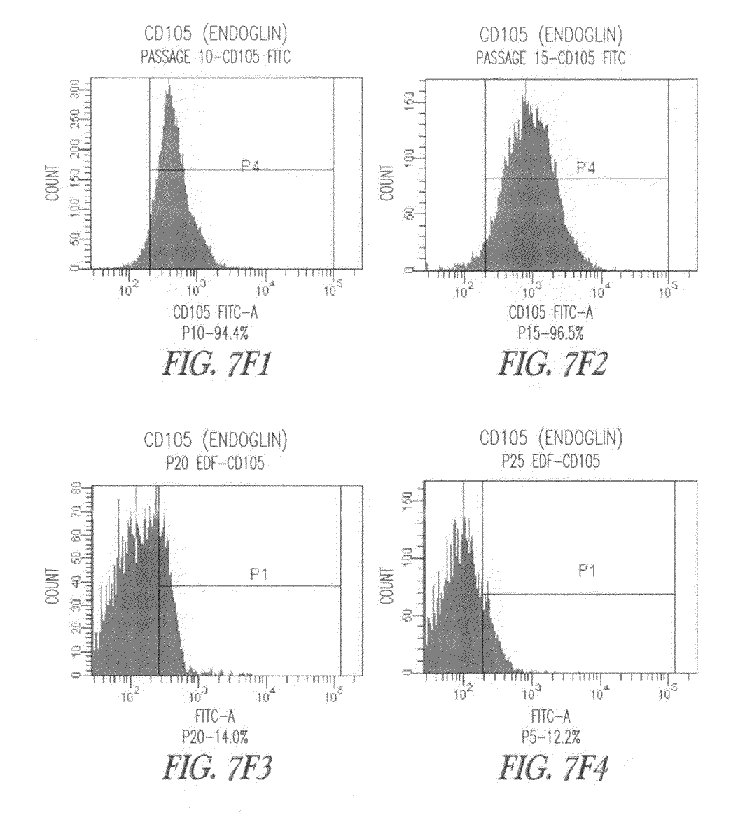

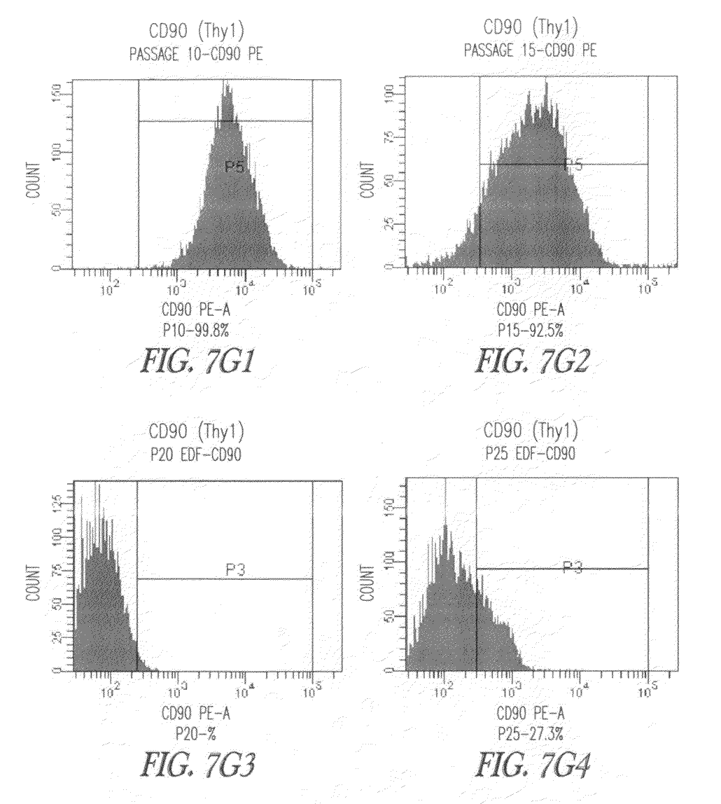

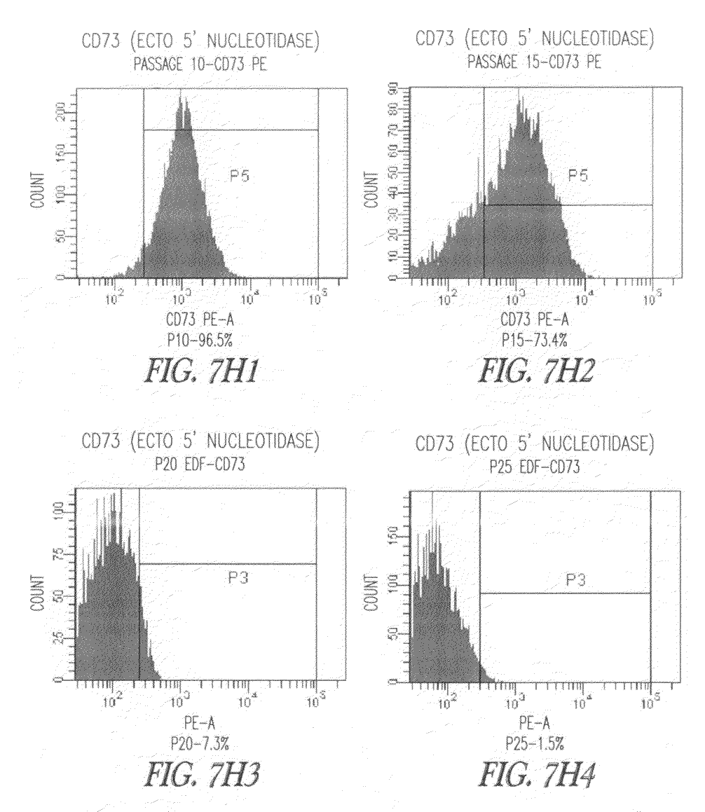

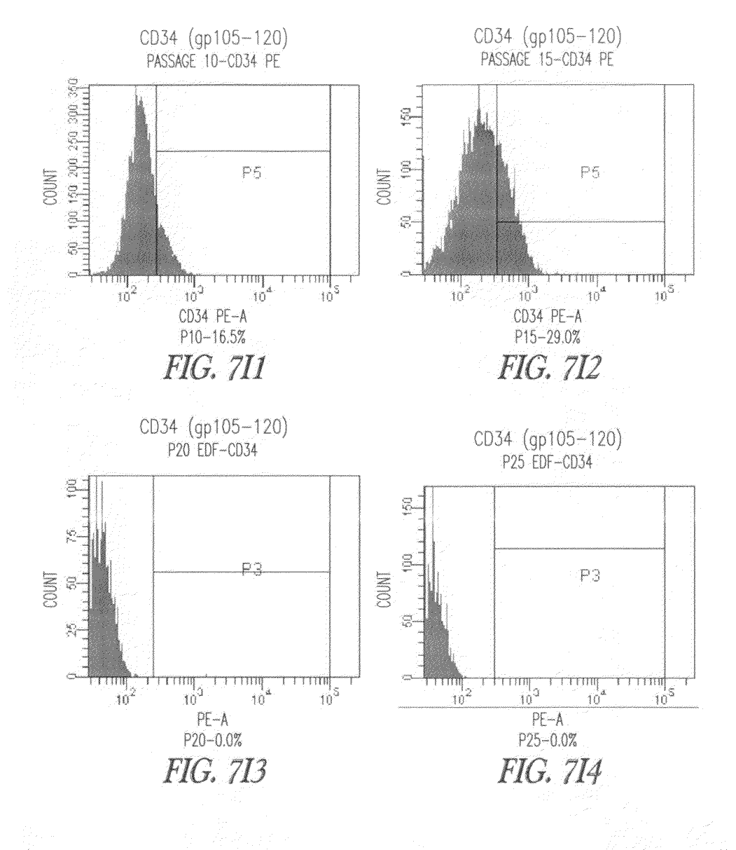

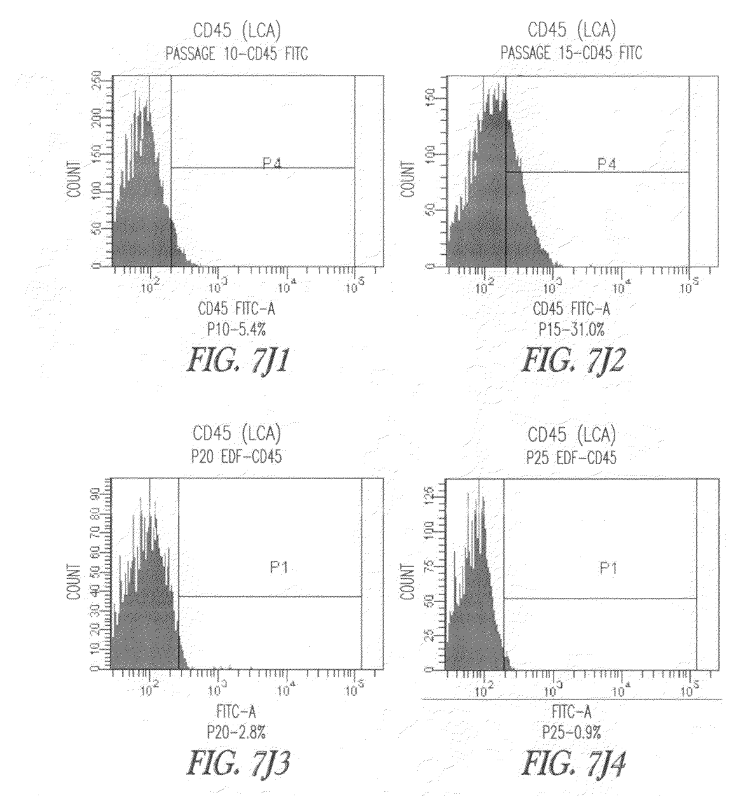

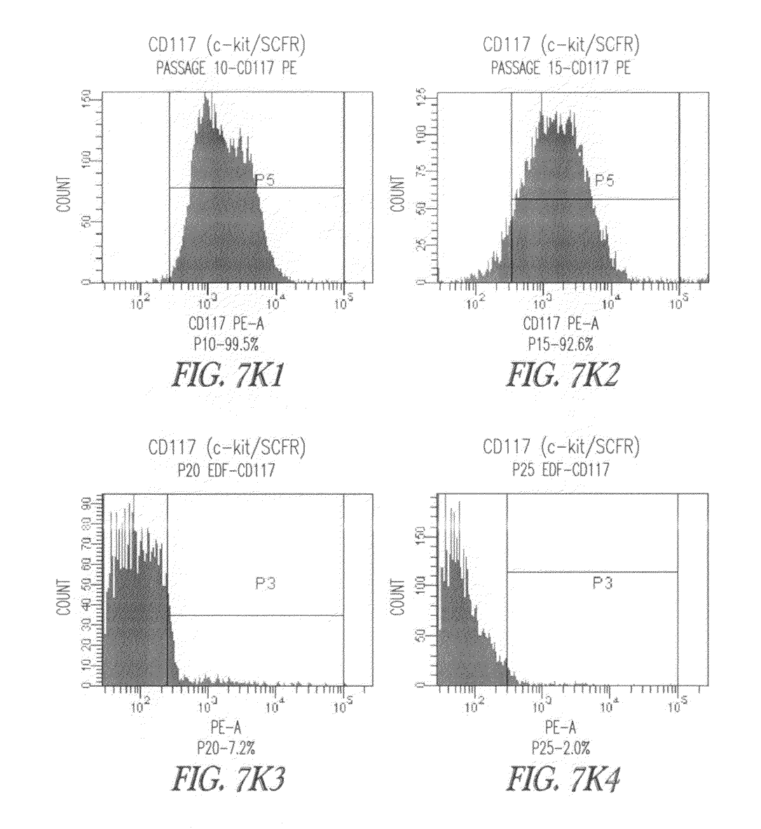

In accordance with present disclosure the human GLDF cells are characterized for specific markers in order to examine the extent of down regulation or up regulation of gene expression profile. Human GLDF cells are characterized by flow cytometry for clusters of differentiation markers (CD)/surface antigens. FIG. 7 shows the expression of cell surface markers at different passages, wherein the markers used for expression profiling of cell surface markers were CD 50, CD 106, CD 44, CD 54, CD 31, CD 105, CD 90, CD 73, CD 34, CD 45, CD 117, and CD 135. It was found that the expression level for these markers increases over passages and later on decreased. Human GLDF cells were found to be highly positive for CD90, CD44 and CD 117, CD73, CD 105 whereas moderately positive for, CD106, CD50, CD54 and CD135 and negative for CD45, CD34, CD31, CD133 (See Table 4). Detailed procedure of the gene expression profile is described in the Example 2.

In one aspect the present disclosure provides undifferentiated, pluripotent and proliferative hESCs cultured on xeno-free culture system comprising human GLDF cells, wherein the hESCs are substantially free of xeno contaminants.

hESC lines co-cultured with human GLDF cells of the present disclosure maintain doubling time of at least 20-25 hours which is faster than the conventional method of using mouse embryonic feeder cells. As observed in this disclosure hESCs maintain in an undifferentiated state for long number of passages.

In preferred embodiments hESCs (HUES-7 and/or HUEC-9) have been cultured on xeno-free culture system comprising GLDF cells, following upon which they are screened for various embryonic and differentiation markers.

In accordance with the present disclosure HUES-7 and HUESC-9 lines were derived from day 5 human embryos obtained after informed consent taken from infertile patients. Institutional Ethics Committee approval was taken before obtaining embryos from the infertile patients. Only spare and supernumerary embryos were taken after the infertility treatment is over. Inner cell mass of the embryos were taken after immunosurgery and cultured on mouse embryonic feeder cells. Both the cell lines were characterized and established for prolonged culture. Method or derivation of HUES-7 and HUES-9 have been described in Example 3.

In an embodiment of the present disclosure HUES-7 cells were thawed and cultured for several passages on a Xeno-free culture system comprising feeder layer of human GLDF cells and a culture medium which further comprises about 70-90% KO-DMEM, about 10-30% human serum, about 2 mM L-glutamine, about 1-2% non-essential amino acids, about 0.1 mM beta-mercaptoethanol and about 4-10 nanogram per milliliter human recombinant basic fibroblast growth factor.

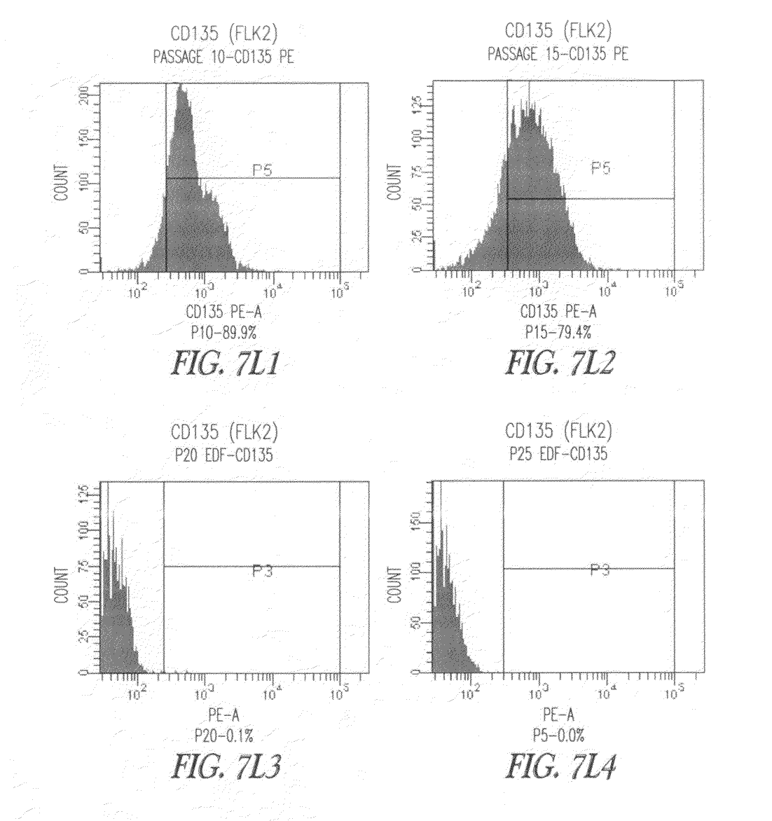

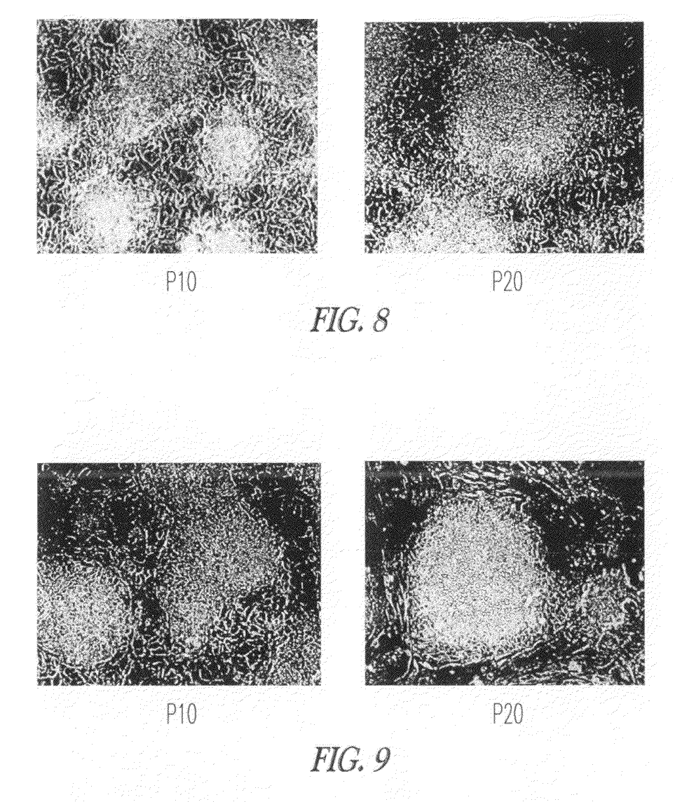

The details of derivation and culture of HUES cell lines are given in Example 3. FIG. 8 shows the morphology of cells of HUES-7 cell lines cultured on human GLDF cells of the present disclosure.

In yet another embodiment of the present disclosure HUES-9 cells were cultured using xeno-free culture system as described in Example 3. FIG. 9 shows the morphology of cells of HUES-9 cell lines cultured on human GLDF cells of the present disclosure.

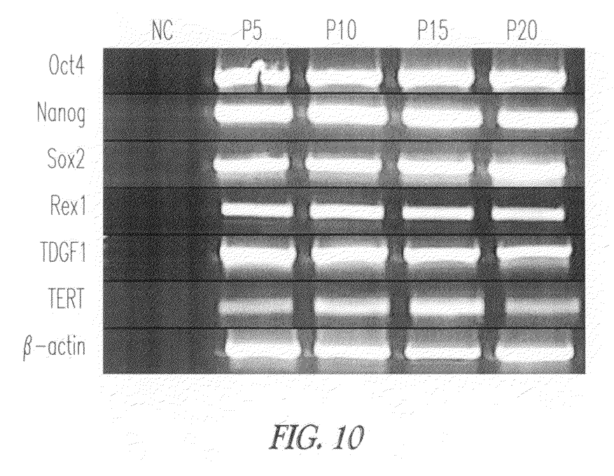

The cultured HUES-7 cells were then characterized for pluripotency by analyzing the presence of pluripotent markers. The cultured cells were found to be undifferentiated and capable of self renewable even after prolonged cultures. It was observed that the cells maintained pluripotency in prolonged, in-vitro culture conditions. FIG. 10 shows RT-PCR results for the expression profiling of pluripotent markers on HUES-7 cells, wherein the markers were OCT-4 Nanog, Sox2, Rex 1, TDGF1, TERT and β-actin (Also see Table 5). GAPDH can also be used as a positive control. The marker specific primers were used in the RT-PCR reaction the nucleotide sequences for which are as shown in SEQ ID NO.: 23-32. The primer sequences used for the expression of GAPDH and OCT-4 are as shown below: and is shown in SEQ ID NO.: 37, 38 and SEQ ID NO.: 39, 40:

| | |
|---|---|
| GAPDH | |
| SEQ ID NO.: 37- | GGGCGCCTGGTCACCAGGGCTG |
| SEQ ID NO.: 38- | GGGGCCATCCACAGTCTTCTG |
| OCT-4 | |
| SEQ ID NO.: 39- | CGACCATCTGCCGCTTTGAG |
| SEQ ID NO.: 40- | CCCCCTGTCCCCCATTCCTA |

Expression profile of pluripotent markers on HUES-7 cells cultured on human GLDF cells was analyzed also by employing immunocytochemistry (See Table 6). Expression of Alkaline phosphatase, OCT-4, SSEA-4 and TRA-1-60 was checked at Passage 20 (P20) in order to confirm the pluripotency capabilities. RT-PCT was also employed to demonstrate expression of pluripotent markers of human embryonic stem cells HUES-7 cultured on GLDF cells. Expression of Oct 4, Nanog, Sox 2, Rex 1, TDGF 1 and TERT was screened at Passage 5 (P5), Passage 10 (P10), Passage 15 (P15), and Passage 20 (P20).

In accordance with the present disclosure the human embryonic stem cells (HUES-7 or HUES-9 as used herein) when co-cultured with the human GLDF cells, were found to remain capable of differentiating into major germ lineages as endoderm, ectoderm, and mesoderm. To confirm the differentiation of hESCs in vitro, feeder free HUES-7 cells were transferred to culture medium comprising 80% KO-DMEM/F, 20% KO-SR, 1 mM L-glutamine, 1% nonessential amino acids, 0.1 mM β-mercaptoethanol except for bFGF and cultured continuously. At specific intervals, total RNA was isolated from cells of EBs using methods known in the art. The differentiation potential of cells was confirmed by performing RT-PCR for various differentiation markers on cells of EBs.

In an embodiment the differentiation markers such as Nestin, NCAM, beta-tubulin, alpha-actinin, myosine heavy chain, brachiury, PDX, alpha fetoprotein, GATA-2, Hand-1, BMP-4 were found negative on cultured HUES-7 cells as screened by methods known in the art. Detailed procedure of the gene expression profile of HUES-7 cells cultured on GLDF cells is described in the Example 4.

Gene expression profiling of the hESCs of HUES-9 cell line was performed using the materials and methods as discussed in example 4. RT-PCR results demonstrated the expression of various pluripotent markers on HUES-9 cultured on GLDF cells. Expression of Oct 4, Nanog, Sox 2, Rex 1, TDGF 1 and TERT was checked at different passages and was found positive.

The present disclosure also provides an alternative method of culture and propagation of hESCs which remain essentially free from feeder cells and xeno-contaminants. Said xeno-free and feeder free culture conditions are achieved by way of conditioned medium derived from GLDF cells. The cultured hESCs remain undifferentiated and capable of differentiating into cells of germ lineages.

The cells used to condition the medium may have one or more desirable features, such as being from a non-malignant source and having a normal karyotype, being capable of extensive culture. The method of producing a conditioned medium further includes validating the ability of the conditioned medium to maintain the stem cells in an undifferentiated state, whereas validating is effected by a differentiation assay selected from the group consisting of morphology analysis, karyotype analysis and surface marker analysis.

One feature in the present disclosure is that the tissue culture medium is a species-derived conditioned medium.

In an embodiment of the disclosure it was determined that high expression of basic fibroblast growth factor in the GLDF cells and so also in conditioned media obtained thereby, which allow derivation, propagation and culture of human embryonic stem cells without addition of basic fibroblast growth factor in the culture medium. Person skilled in the art may know that basic fibroblast growth factor is a key component to keep the hESCs pluripotent and self renewal.

In accordance with the present disclosure the conditioned medium is derived from human GLDF cells. In particular aspect, the mitotically inactivated human GLDF cells are cultured for 24-48 hours on a growth medium comprising of DMEM high glucose, 20% human serum, 2 mM L-glutamine, 2% non-essential amino acids, 0.1 mM beta-mercaptoethanol and 4 ng/ml human recombinant basic fibroblast growth factor. The supernatant is then collected and used as conditioned medium.

In accordance with the present disclosure there is provided a method of culturing hESCs in a growth environment essentially free from feeder cells comprising conditioned medium which further comprises of essential media supplements. HUES-7 and/or HUES-9 cells were cultured using conditioned medium.

In preferred embodiment the HUES-7 cells are cultured on Petri dish coated with extra cellular matrix selected from the group consisting of fibronectin, laminin, collagen IV, collagen III, gelatin and a combination thereof.

An embodiment of the present disclosure relates to extra cellular matrix is selected from the group consisting of fibronectin, laminin, collagen IV, collagen III, gelatin and a combination thereof.

In a preferred embodiment the extra-cellular matrix is fibronectin which is further selected from the group consisting of human fibronectin, recombinant human fibronectin, human collagen, human laminin, synthetic fibronectin and a combination thereof. According to still further features in the described preferred disclosure the matrix is a species-derived fibronectin matrix.

In accordance with the present disclosure the HUES-7 cells obtained by culturing in feeder free cultures are maintainable in an undifferentiated, pluripotent and proliferative state for about 30-60 passages and at least 50 passages. Further, the cultured HUES-7 cells maintain a doubling time of at least 20 to 25 hours. Similarly, HUES-9 cells were also cultured using method described in the disclosure and were found to maintain pluripotency for several passages. Detailed procedure of the culturing and propagating HUES-7 and HUES-9 on conditioned medium is described in the Example 6.

In accordance with the present disclosure the hESCs obtained by culturing on conditioned medium are characterized for various pluripotent and differentiated markers.

Human embryonic stem cells were analyzed for pluripotent markers such as OCT-4, NANOG, REX-1, TDGF, SOX-2 and TERT (GAPDH as housekeeping genes). Cultured cells of both HUES-7 and HUES-9 lines were screened for pluripotency and the expression of the above markers were confirmed by RT-PCR. Primers sequences as shown in SEQ ID NOs.: 23-32 and SEQ ID NOs.: 37-40 were employed for the assay. RT-PCR was carried out for expression of pluripotent markers in HUES-7 and HUES-9 cultures using conditioned medium from GLDF at passage 5 (P5). Expression of Oct4-lane2, Nanog-lane3, Sox2-lane4, Rex1-lane5, TDGF1-lane6, and TERT-lane7 was screened against (3-actin-lane1 which was used as negative control. (See Table 7)

Expression of surface markers on HUES-7 and HUES-9 cultured using feeder free conditioned medium obtained from GLDF cells was screened using Immunocytochemistry. The results demonstrate alkaline phosphatase activity and expression of pluripotency markers. The markers screened were Alkaline Phosphatase, SSEA-3, SSEA-4, TRA-1-60, TRA-1-81, and SOX-2. (Also see Table 8)

Pluripotency of hESCs was also analyzed by flow cytometry by screening the expression of SSEA1, SSEA4, TRA1-60, TRA1-81. The expression of markers on HUES-7 and HUES-9 cells cultured on matrigel using conditioned medium from GLDF at passage 10 was demonstrated.

Differentiation markers such as Nestin, NCAM, beta-tubulin, alpha-actinin, myosine heavy chain, brachiury, PDX, alpha fetoprotein, GATA-2, Hand-1, BMP-4 were found negative on cultured human ES cell lines as screened by method known in the art. Detailed procedure of the gene expression profile is described in the Example 7.

### EXAMPLES

It should be understood that the following examples described herein are for illustrative purposes only and that various modifications or changes in light will be suggested to persons skilled in the art and are to be included within the spirit and purview of this application and the scope of the appended claims

### Example 1

### Generation of Germ Lineage Derived Feeder Cells (GLDF Cells)

### Direct Differentiation to Obtain Embryoid Bodies

Embryoid bodies (EBs) were obtained by culturing hESCs in suspension for 7 days. hESCs were harvested by using 0.05% trypsin (Invitrogen) and plated on non-tissue culture treated dishes (approximately 10⁷ cells/10 cm dish), and grown in medium for 7 days. Media comprises of KO-DMEM basal medium supplemented with 20% human serum, glutamine, 1% non-essential amino acid, beta mercaptoethanol and pen-strep. The number of EBs was determined by counting EBs in 20 different fields at a low magnification (10×) using an TE2000 microscope (Nikon). Media was changed after 3 days.

### Obtaining Germ Lineage Derived Feeder Cells (GLDF Cells)

To prepare hESC-derived feeders or the GLDF cells, EBs were plated in a T75 tissue culture flask coated with 0.1% gelatin in a GLDF media which consists of KO-DMEM supplemented with 10% KO-Serum or 10% human serum, 2 mM Glutamine, 1×10⁻⁸ M dexamethasone, 1× insulin-transferrin-selenium and 10 ng/ml epidermal growth factor. After 10 days, differentiated cells were digested with 0.05% trypsin/0.53 mM EDTA and split into two flasks (passage 1 [P1]). After 3-5 days, when cells reached 90% confluence, cells were again split to obtain Passage 2 [P2] cells. Cells of P5 and after were used as feeders and were named as GLDF feeders. For derivation and long-term culture of hESCs, cultured GLDF feeders were mitotically inactivated with 10 mg/ml mitomycin C for 2.5 h and washed three times with PBS. Mitotically inactivated GLDF were then trypsinized with trypsin-EDTA and washed twice with culture medium. The dissociated GLDF were counted and plated on gelatin-coated 35 mm dish plates at 8.0×10⁵ cells per plate. (See FIG. 1)

### Example 2

### Gene Expression Profile

### Characterization of GLDF Cells for Differentiation Markers by RT-PCR

Cells were analyzed for the differentiation markers after different passages. GLDF cells were analyzed for the expression of differentiation markers by RT-PCR. GLDF cells were positive for the expression of Nestin, NCAM, tubulin, GATA2, GATA-4, BMP2, BMP4, Hand1, Vimentin, CK18, CK19, NF heavy chain, NF light chain and GFAP.

RNA extractions were carried out with the RNeasy mini kit. GLDF were vortexed for 1 min to shear genomic DNA before loading onto the columns, and then eluted in a minimum volume of 30 μl and a maximum volume of 2×50 μl RNAse-free water. RNA obtained with this procedure was essentially free of genomic DNA. When using different extraction procedures, a DNAse I treatment, followed by phenol extraction and ethanol precipitation, was applied to remove traces of contaminating DNA.

RNA obtained from the cells was reverse transcribed in the presence of 5 mM MgCl₂, 1×PCR Buffer II, 1 mM dNTPs, 25u MuLV Reverse Transcriptase, 1u RNA inhibitor, 2.5 μM Random hexamers in a final reaction volume of 20 Reactions were carried out at 42° C. for 30 minutes in a thermocycler, followed by a 10 minute step at 99° C., and then by cooling to 4° C. 2 μl of cDNA products were amplified with 1 unit of Taq polymerase in the buffer provided by the manufacturer which contains no MgCl₂, and in the presence of the specific primers having nucleotide sequence as shown in SEQ ID NOs.: 1-20 together with the beta-actin primers (SEQ ID NO.: 21 and SEQ ID NO.: 22) used as an internal control. The amount of dNTPs carried over from the reverse transcription reaction is fully sufficient for further amplification. A first cycle of 10 minutes at 95° C., 45 seconds at 65° C. and 1 minute at 72° C. was followed by 45 seconds at 95° C., 45 seconds at 65° C. and 1 minute at 72° C. for 30 cycles. The conditions were chosen so that none of the RNAs analyzed reached a plateau at the end of the amplification protocol, i.e. they were in the exponential phase of amplification, and that the two sets of primers used in each reaction did not compete with each other. Each set of reactions always included a no-sample negative control.

The PCR products were loaded onto ethidium bromide stained 1 to 2% (depending on the size of the amplification products) agarose gels in TBE. A 100 by DNA ladder molecular weight marker was run on every gel to confirm expected molecular weight of the amplification product.

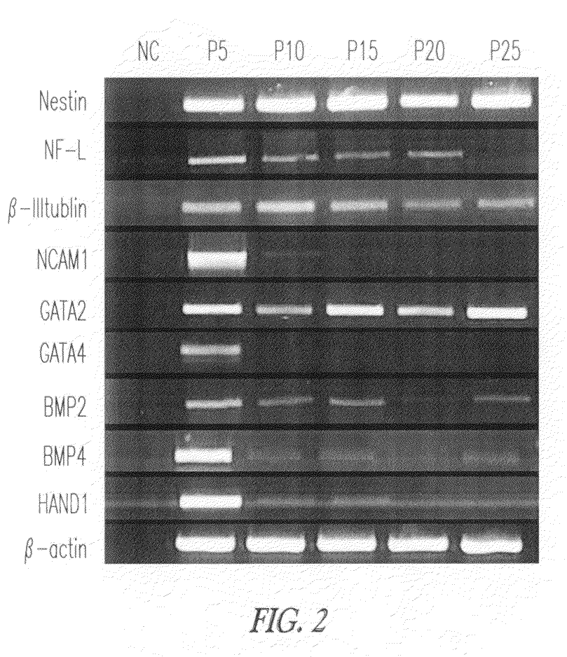

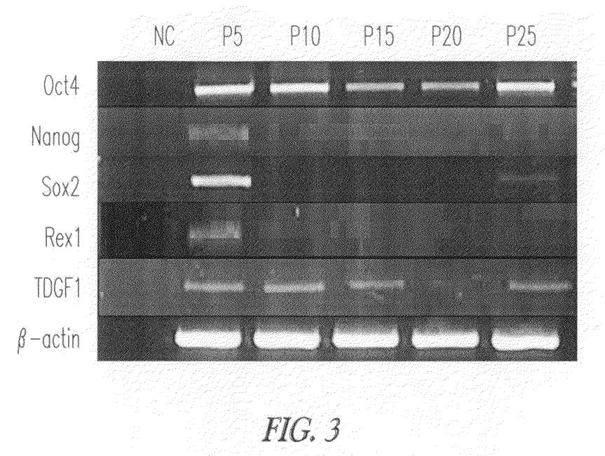

Images of the RT-PCR ethidium bromide-stained agarose gels were acquired with a gel documentation system and quantification of the bands was performed. Band intensity was expressed as relative absorbance units (See FIG. 2). The ratio between the sample RNA to be determined and control (Beta-Actin) was calculated to normalize for initial variations in sample concentration and as a control for reaction efficiency. Mean and standard deviation of all experiments performed were calculated after normalization to beta-Actin. Results are provided in Table 1. (Refer FIG. 2)

TABLE 1
Analysis of Markers on GLDF cells

| Markers | GLDF |
|---|---|
| Vimentin | Positive |
| Nestin | Positive |
| NF light chain | Positive |
| NCAM | Positive |
| GFAP | Positive |
| β-III tubulin | Positive |
| GATA2 | Positive |
| GATA-4 | Positive |
| BMP2 | Positive |
| BMP4 | Positive |
| Hand1 | Positive |

### Characterization of GLDF Cells for Pluripotent Markers by RT-PCR

Cells were analyzed for the pluripotent markers after passage 4. GLDF cells were analyzed for the expression of pluripotent markers by RT-PCR. GLDF cells were negative for NANOG, SOX-2, REX-1, TDGF-1 and TERT. This clearly showed that GLDF cells lost embryonic like properties and become differentiated cells.

RT-PCR reaction was carried out as described above. The reaction was carried out in the presence of the specific primers having nucleotide sequence as shown in SEQ ID NOs.: 23-32 together with the beta-actin primers (SEQ ID NO.: 21 and SEQ ID NO.: 22) used as an internal control.

Images of the RT-PCR ethidium bromide-stained agarose gels were acquired with a gel documentation system (See FIG. 3) and quantification of the bands was performed.

TABLE 2
Analysis of Pluripotent Markers on GLDF cells (By RT-PCR)

| Pluripotent Markers | GLDF |
|---|---|
| β-actin control | Positive |
| Nanog | Negative |
| Sox2 | Negative |
| Rex1 | Negative |
| TDGF1 | Negative |
| TERT | Negative |

### Characterization of GLDF Cells for Fibroblast Markers by RT-PCR

Similarly characterization of GLDF Cells for the expression of fibroblast markers was carried out using RT-PCR. The markers considered for characterization were Vimentin, P4Hβ and bFGF.

RT-PCR reaction was carried out as described above. The reaction was carried out in the presence of the specific primers for Vimentin and P4Hβ (SEQ ID NOs: 17, 18, 33 and 36) together with the beta-actin primers (SEQ ID NOs 21 and 22). Expression of beta-actin was again used as an internal control.

Images of the RT-PCR ethidium bromide-stained agarose gels were acquired with a gel documentation system (See FIGS. 4 and 5) and quantification of the bands was performed.

TABLE 3
Analysis of fibroblast markers on GLDF cells (By RT PCR)

| Fibroblast Markers | GLDF |
|---|---|
| Vimentin | Positive |
| P4Hβ | Positive |
| bFGF (217 bp) | Positive |

### Characterization of GLDF Cells by Immunocytochemistry

GLDF cells were fixed in 4% paraformaldehyde in phosphate buffered saline, 0.05% Triton X-100 for 30 minutes at room temperature and incubated with primary antibodies overnight at 4° C. Fluorescein isothiocyanate (FITC)-conjugated secondary antibodies (1:100); antibodies against Vimentin, Nestin and P4HB were used for the expression profiling. The specificity of each antibody was verified by negative controls included in each experiment. The slides were analyzed using inverted microscope. (See FIG. 6)

### Characterization of GLDF Cells for Differential Markers by Flow Cytometry

Characterization of cell surface cluster differentiation (CD) markers on GLDF cells to aid in analyzing the expression of cell surface markers was done. Flow cytometry showed cell populations positive for CD44, CD50, CD54, CD73, CD90, CD105, CD106, CD117 and CD135, and negative for CD31, CD34, CD45, CD133.

Aliquots of GLDF cells were allowed to expand at 37° C. and 95% air/5% CO2 humidified environment. After expansion, cells were dissociated with 0.05% trypsin-EDTA and re-suspended in buffer. The cells were then centrifuged and re-suspended in wash buffer at a concentration of 1×10⁶ cells/ml. Wash buffer consisted of phosphate buffer supplemented with 1% (v/v) FBS and 1% (w/v) sodium azide. Cell viability was >98% by the Trypan blue exclusion method. 100 μl of cell preparation 1×10⁵ were stained with saturating concentrations of fluorescein isothiocyanate-(FITC), phycoerythrin-(PE), conjugated markers and isotype matched controls. Briefly, cells were incubated in the dark for 30 min. at 4° C. After incubation, cells were washed three times with wash buffer and resuspended in 0.5 ml of wash buffer for analysis on the flow cytometer. Flow cytometry was performed on a LSR-II. Cells were identified by light scatter. Logarithmic fluorescence was evaluated (4 decade, 1024 channel scale) on 10,000 gated events. Analysis was performed using software known in the art and the presence or absence of each antigen was determined by comparison to the appropriate isotype control. An antigenic event was observed when the fluorescence was greater than 25% above its isotype control. Statistical analysis was performed on the pooled flow cytometric data from the three mesenchymal stem cell lines. Thus, a sample size of three was used for each CD marker. A mean value above 1000 cells was considered positive for any CD marker. Results are given in Table-6. (Also see FIG. 7)

TABLE 4
Analysis of Cluster of Differential Markers on GLDF cells

| Differential | | |
|---|---|---|
| Markers | Results | Percentage |
| CD90 | Positive | 92.5% |
| CD105 | Positive | 96.5% |
| CD73 | Positive | 73.4% |
| CD45 | Negative | 31.0% |
| CD34 | Negative | 29.0% |
| CD44 | Positive | 82.3% |
| CD106 | Positive | 61.6% |
| CD31 | Negative | 11.7% |
| CD50 | Negative | 27.7% |
| CD54 | Positive | 92.6% |
| CD133 | Negative | 5.82% |
| CD117 | Positive | 92.6% |
| CD135 | Positive | 79.4% |

### Karyotyping:

It has been reported that karyotype instability can sometimes be observed with long-term passages of cells. In order to determine the karyotypic instability, karyotyping of the GLDF cells was done at different stages, preferably after every 10 passages. GLDF cells were grown in 60 mm plate on high density. Colcemid solution was added on the following day directly into the plate at the final concentration of 0.02 μg/ml. Cells were incubated for 2 hours at 37° C. and 5% CO₂. Culture media containing colcemid was removed after the incubation was over and cells were dissociated with 0.05% trypsin free from EDTA. Cells were transferred into 15 ml tube and 10 ml FBS in DMEM-F-12 was added. Cells were washed by centrifuging at 1000 rpm for 5 minutes at room temperature. Supernatant was removed and re-suspend the pellet in 2 ml of warm hypotonic solution. Cells were mixed properly and incubated in a water bath at 37° C. for 30 minutes. 0.5 ml of fixative is added drop-wise with swirling. Cells were centrifuged again at 1000 rpm for 5 minutes at room temperature. Supernatant was aspirated and 1 ml of fixative was added drop-wise while swirling the cells. This was done at least 2 times.

To make the spread, surface of the slide is humidified by application of warm breath whilst holding the slide at a 45° angle. One drop of the suspended cells is carefully dropped from the height of approximately 0.5 meter using Pasteur pipette onto the top surface of the slide and it was allowed to air dry. Slide was stained with freshly made Leishman's stain for 8 minutes and was rinsed in running water for 1 minute and air dried. Cells were mounted with coverslip using depex. Karyotyping of GLDF cells maintained in culture until passage 25 was found to be normal

### Example 3

### Culture and Propagation of Human Embryonic Stem Cells Using GLDF Cells

### Derivation of Human Embryonic Stem Cell Lines (HUES-7 and HUES-9)

Human embryos were produced by the ART Center, Manipal Hospital, Bangalore. Surplus embryos were used for hESC derivation with informed consent. The procedure to derive hESCs from surplus embryos was in accordance with the Guidelines of Indian Council of Medical Research (ICMR) and approved by the Ethics Committee of Manipal Hospital.

Zona pellucida of the blastocyst was removed with 0.5% pronase. Inner cell mass was isolated manually and cultured on xeno-free culture system comprising Mit-C treated GLDF feeder cells prepared as described above. The culture medium consisted of 78% KO-DMEM/F, 20% KO-SR, 2 mM L-glutamine, 1% nonessential amino acids, 0.1 mM β-mercaptoethanol, and 4 ng/ml bFGF. The medium was changed every day. Ten to 14 days after initial plating, colonies with typical hESCs morphology appeared. These colonies were dissociated mechanically and transferred onto a fresh dish with human GLDF cells.

### Culture and Propagation of Human Embryonic Stem Cell Lines

HUES-7 and HUES-9 cells has been cultured using xeno-free culture system comprising human GLDF cells. However, hESCs obtained from various sources can be cultured and propagated using GLDF cells. Human ES cells (HUES-7 or HUES-9) were trypsinized with trypsin-EDTA and washed twice with media and transferred to culture dishes preplated with Human GLDF cells. Long-term culture of hESCs was performed by passaging hESCs every 5-6 days using trypsin in combination with manual dissociation. hESCs were cryopreserved in freezing media consisting of 90% KO-SR and 10% dimethylsulfoxide. To determine population doubling (PD) time, cell numbers in five selected independent colonies were counted under an inverted microscope. Data collected on days 1 and 2 (with 36 hours apart) were used to calculate PD values: PD=log 2, in which N1 and N2 are the cell numbers of selected colonies counted on day 1 and day 2, respectively. See FIGS. 8 and 9 for the morphology of HUES-7 and HUES-9 cells cultured on human GLDF cells.

### Example 4

### Gene Expression Profiling of hESCs

### Characterization of hES Cells for Pluripotent Markers by RT-PCR:

HUES-7 cells were analyzed for the expression of pluripotent markers at passage 4 by RT-PCR and were positive for OCT-4, Nanog, as compared with the expression of Beta actin marker which was used as positive control.

RT-PCR reaction was carried out as described above. The reaction was carried out in the presence of the specific primers. Primer sequences for OCT-4, Nanog, Rex-1, TDGF, TERT, and SOX-2 are as shown in SEQ ID NOs.: 23-32 and SEQ ID NOs.: 39 and 40. The expression of GAPDH marker can also be used as an internal control, the primer sequences in that case will be as shown in SEQ ID NO.: 37 and 38.

Images of the RT-PCR ethidium bromide-stained agarose gels were acquired with a gel documentation system (See FIG. 10) and quantification of the bands was performed.

TABLE 5
Primer Sequences Used in PCR:

| Primers | Size | Results |
|---|---|---|
| OCT-4 | 572 | Positive |
| Nanog | 262 | Positive |
| Rex-1 | 303 | Positive |
| TDGF1 | 498 | Positive |
| SOX2 | 448 | Positive |
| TERT | 602 | Positive |
| GAPDH | 564 | Positive |
| | | control |

### Characterization of Human Embryonic Stem Cell Lines by Immunocytochemistry

Immunocytochemistry was performed as explained above in Example 2. Fluorescein isothiocyanate (FITC)-conjugated secondary antibodies (1:100) against SSEA-1 (1:100), SSEA-3 (1:200), SSEA-4 (1:200), TRA-1-60 (1:100), and TRA-1-81 (1:100), Sox-2 and alkaline phosphatase were used. The results are given below in Table 8.

TABLE 6
Analysis of Markers on hESCs (By Immunocytochemistry)

| | Human ES |
|---|---|
| Markers | Cells |
| SSEA-1 | Negative |
| SSEA-3 | Positive |
| SSEA-4 | Positive |
| SOX-2 | Positive |
| Alkaline Phosphatase | Positive |
| TRA-1-60 | Positive |
| TRa-1-81 | Positive |

### Example 5

### Conditioned Medium

Conditioned media contain secreted growth factor from the cells that could be used for growing cells without directly using feeder cells. In order to obtain xeno-free and feeder free human embryonic stem cell lines Conditioned media could be used. In the present disclosure GLDF cells were cultured in the GLDF medium of for 24 hours. Next day media was changed from GLDF medium to ES cell medium. Cells were cultured from another 48 hours in ES cell medium. After 48 hours of culture supernatant was removed and filtered through 0.22 micron low protein binding filter and used for ES cell derivation, propagation and culture.

Initially human embryonic stem cells were differentiated into Embryoid bodies (EBs) and were plated in a T75 tissue culture flask coated with 0.1% gelatin in a GLDF media consists of KO-DMEM supplemented with 10% KO-Serum Or 10% human serum, 1% Gulamine, 1×10⁻⁸ M dexamethasone, 1× insulin-transferrin-selenium and 10 ng/ml epidermal growth factor. After 10 days, differentiated cells were digested with 0.25% trypsin/0.53 mM EDTA and split into two flasks (passage 1 [P1]). After 3-5 days, when cells reached 90% confluence, cells were again split to obtain P2 cells. Cells of P5 and after were used as feeders and were named GLDF feeders. Derivation and long-term culture of hESCs, cultured GLDF feeders were mitotically inactivated with 10 mg/ml mitomycin C for 2.5 h and washed three times with PBS. Mitotically inactivated GLDF were then trypsinized with trypsin-EDTA and washed twice with culture medium. The dissociated GLDF were counted and plated on gelatin-coated 35 mm dish plates at 8.0×10⁵ cells per plate. Upon 80% confluency GLDF medium was removed and replaced with ES medium that consists of DMEM high glucose, 20% human serum, 2 mM L-glutamine, 2% non-essential amino acids, 0.1 mM beta-mercaptoethanol and with or without 4 nanogram per milliliter human recombinant basic fibroblast growth factor. After 48 hours of culture the media was removed and used as a conditioned media for subsequent use for culturing human embryonic stem cells.

### Example 6

### Culture and Propagation of Human Embryonic Stem Cell Lines

The cells of HUES-7 and HUES-9 were cultured on conditioned medium by employing method as described in Example 3.

### Example 7

### Gene Expression Profiling of hESCs Cultured on Conditioned Medium

### Characterization of ES Cells for Pluripotent Markers by RT-PCR:

HUES-7 and HUES-9 cells cultured on conditioned medium were analyzed for various pluripotent markers employing RT-PCR (See Example 4). The results are shown below in Table 8.

TABLE 7
Analysis of Pluripotent Markers on hESCs
cultured on conditioned medium:

| Primers | Size | Results |
|---|---|---|
| OCT-4 | 572 | +ve |
| Nanog | 262 | +ve |
| Rex-1 | 303 | +ve |
| TDGF1 | 498 | +ve |
| SOX2 | 448 | +ve |
| TERT | 602 | +ve |
| Beta-actin | 353 | +ve |
| | | control |

### Characterization of Human Embryonic Stem Cell Lines by Immunocytochemistry

Immunocytochemistry was performed as explained in Example 4. Fluorescein isothiocyanate (FITC)-conjugated secondary antibodies (1:100) against SSEA-1 (1:100), SSEA-3 (1:200), SSEA-4 (1:200), TRA-1-60 (1:100), and TRA-1-81 (1:100), Sox-2 and alkaline phosphatase were taken. Results are given below in Table 9.

TABLE 8
Analysis of Pluripotent Markers on hESCs cultured on
Conditioned medium as screened by Immunocytochemistry

| Markers | Human ES Cells |
|---|---|
| SSEA-1 | Negative |
| SSEA-3 | Positive |
| SSEA-4 | Positive |
| SOX-2 | Positive |
| Alkaline Phosphatase | Positive |
| TRA-1-60 | Positive |
| TRa-1-81 | Positive |

### Characterization of Human Embryonic Stem Cell Lines by Flow Cytometry

The hESCs (HUES-7 and HUES-9) cultured on conditioned medium of the disclosure were analyzed for the expression of pluripotency markers SSEA1, SSEA4, TRA1-60 and TRA1-81 on HUES7 and HUES9 at passage10, cultured on matrigel using GLDF-CM. The method is as described in Example 2.

The above analysis indicate that Xeno-free culture system comprising human GLDF cells and conditioned medium obtained from human GLDF cells can be used as suitable medium for derivation, culture and propagation of hESCs, wherein the hESCs are maintained in pluripotent state for several passages.

### BIBLIOGRAPHY

Amit M, Margulets V, Segev H et al. Human feeder layers for human embryonic stem cells. Biol Reprod 2003; 68:2150-2156.
Amit M, Shariki C, Margulets V et al. Feeder layer- and serum-free culture of human embryonic stem cells. Biol Reprod 2004; 70:837-845.
Amit M, Winkler M E, Menke S, Bruning E, Buscher K, Denner J, Haverich A, Itskovitz-Eldor J, Martin U. No evidence for infection of human embryonic stem cells by feeder cell-derived murine leukemia virus. Stem Cell 2005. 23:761-771.
Cheng L, Hammond H, Zhaohui Y et al. Human adult marrow cells support prolonged expansion of human embryonic stem cells in culture. Stem Cells 2003; 21:131-142.
Evans M J, Kaufman M H. Establishment in culture of pluripotential cells from mouse embryos. Nature. 1981 Jul. 9; 292(5819):154-6.
Graves K H, Moreadith R W Derivation and characterization of putative pluripotential embryonic stem cells from preimplantation rabbit embryos. Mol Reprod Dev. 1993 December; 36(4):424-33
Heins N, Englund M C, Sjoblom C, Dahl U, Tonning A, Bergh C, Lindahl A, Hanson C, Semb H. Derivation, characterization, and differentiation of human embryonic stem cells. Stem Cells. 2004; 22(3):367-76.
Iannaccone P M, Taborn G U, Garton R L, Caplice M D, Brenin D R. Pluripotent embryonic stem cells from the rat are capable of producing chimeras. Dev Biol. 1994 May; 163(1):288-92.
Lee J B, Song J M, Lee J E, Park J H, Kim S J, Kang S M, Kwon J N, Kim M K, Roh S, Yoon H S. Available human feeder cells for the maintenance of human embryonic stem cells. Reproduction 2004. 128: 727-735
Martin G R. Isolation of a pluripotent cell line from early mouse embryos cultured in medium conditioned by teratocarcinoma stem cells. Proc Natl Acad Sci USA. 1981 December; 78(12):7634-8.
Martin M J, Muotri A, Gage F, Varki A. Human embryonic stem cell express an immunogenic non-human sialic acid. Nat Med. 2005. 11: 228-232.
Notarianni E, Galli C, Laurie S, Moor R M, Evans M J. Derivation of pluripotent, embryonic cell lines from the pig and sheep. Department of Genetics, University of Cambridge, UK. J Reprod Fertil Suppl. 1991; 43:255-60.
Reubinoff B E, Pera M F, Vajta G et al. Effective cryopreservation of human embryonic stem cells by the open pulled straw vitrification method. Hum Reprod 2001; 16:2187-2194.
Richards M, Tan S, Fong C-Y et al. Comparative evaluation of various human feeders for prolonged undifferentiated growth of human embryonic stem cells. Stem Cells 2003; 21: 546-556.
Richards M, Fong C Y, Chan W K, Wong P C, Bongso A. (2002). Human feeders support prolonged undifferentiated growth of human inner cell masses and embryonic stem cells. Nat. Biotechnol. 20: 933-6)
Sato N, Meijer, L, Skaltsounis L, Greengard P, Brivanlou A H. Maintenance of pluripotency in human and mouse embryonic stem cells through activation of Wnt signaling by a pharmacological GSK-3-specific inhibitor. Nat. Med. 2004 January; 10(1):55-63. Epub 2003 Dec. 21.
Shamblott M J, Axelman J, Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci USA. 1998 Nov. 10; 95(23):13726-31. Erratum in: Proc Natl Acad Sci USA 1999 Feb. 2; 96(3):1162.
Thomson J A, Kalishman J, Golos T G, Durning M, Harris C P, Becker R A, Hearn J P. Isolation of a primate embryonic stem cell line. Proc Natl Acad Sci USA. 1995 Aug. 15; 92(17):7844-8.
Thomson, J. A., Itskovitz-Eldor, J., Shapiro, S., Waknitz, M. A., Sweirgiel, J. J., Marshall, V. S., et al. (1998). Embryonic stem cell lines derived from human blastocysts. Science, 282, 1145-1147
Xu C, Inokuma M S, Denham J et al. Feeder-free growth of undifferentiated human embryonic stem cells. Nat Biotechnol 2001; 19: 971-974.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A Xeno-free culture system for culturing human embryonic stem cells (hES), wherein the culture system comprises:
(a) human germ lineage derived feeder cells (GLDF cells);
(b) culture medium supplemented with growth factors, serum supplements, media supplements or a combination thereof.

2. The Xeno-free culture system as claimed in claim 1, wherein the GLDF cells are derived from human embryonic stem cell lines.

3. The Xeno-free culture system as claimed in claim 1, wherein the GLDF cells are fibroblast like cells.

4. The Xeno-free culture system as claimed in claim 1, wherein the growth factors in
the culture medium are selected from the group consisting of transforming growth factor-β-1 (TGF-β-1), epidermal growth factor (EGF), Activin-A, Activin-B, Acidic FGF, brain derived neurotrophic factor (BDNF), platelet derived growth factor (PDGF), human Insulin growth factor (IGF), Keratenocyte growth factor (KGF), stem cell factor (SCF), bone morphogenic protein (BMP4), hepatocyte growth factor (HGF), nerve growth factor (βNGF), Insulin, selenite, transferrin, Neurotropin 3 (NT3), Neurotrophin 4 (NT4), N2B27 and a combination thereof.

5. The culture medium as claimed in claim 4, wherein the TGF-β-1 is provided at a concentration of about 1-20 ηg/ml.

6. The culture medium as claimed in claim 4, wherein the EGF is provided at the concentration of about 1-20 ng/ml.

7. The culture medium as claimed in claim 4, wherein the activin A is provided at the concentration of about 5-100 ng/ml.

8. The culture medium as claimed in claim 4, wherein the activin B is provided at the concentration of about 5-100 ng/ml.

9. The culture medium as claimed in claim 4, wherein the acidic FGF is provided at the concentration of about 1-20 ng/ml.

10. The culture medium as claimed in claim 4, wherein the BDNF is provided at the concentration of about 1-20 ng/ml.

11. The culture medium as claimed in claim 4, wherein the PDGF is provided at the concentration of about 1-20 g/ml.

12. The culture medium as claimed in claim 4, wherein the IGF is provided at the concentration of about 2-20 ng/ml.

13. The culture medium as claimed in claim 4, wherein the KGF is provided at the concentration of about 10-50 ng/ml.

14. The culture medium as claimed in claim 4, wherein the SCF is provided at the concentration of about 5-20 ng/ml.

15. The culture medium as claimed in claim 4, wherein the BMP4 is provided at the concentration of about 5-20 ng/ml.

16. The culture medium as claimed in claim 4, wherein the HGF is provided at the concentration of about 10-20 ng/ml.

17. The culture medium as claimed in claim 4, wherein the NGF is provided at the concentration of about 20-100 ng/ml.

18. The Xeno-free culture system as claimed in claim 1, wherein the culture medium comprises about 70-90% KO-DMEM, about 10-30% human serum, about 1-2 mM L-glutamine, about 1-2% non-essential amino acids, about 0.1 mM beta-mercaptoethanol and about 4-8 nanogram per milliliter human recombinant basic fibroblast growth factor.

19. A method of derivation and culturing human embryonic stem cells (hESCs), wherein said method comprises culturing the hESCs on Xeno-free culture system of claim 1.

20. A method as claimed in claim 19, wherein the hESCs remain capable of differentiation into ectoderm, mesoderm and endoderm lineages.

21. The method as claimed in claim 19, wherein the hESCs are maintained in proliferative and undifferentiated state for about 30-60 passages, preferably about 35 passages.

22. The method as claimed in claim 19, where hESCs are cultured in Petri dish coated with extra cellular matrix selected from a group comprising of Matrigel, fibronectin, poly-L-lysine, laminin, collagen IV, collagen III and a combination thereof.

23. A conditioned medium for culturing human embryonic stem cells (hESCs), said medium prepared by the method comprising:
(a) culturing GLDF cells for 24 hours on a growth medium comprising DMEM high glucose, about 20% human serum, about 2 mM L-glutamine, about 2% non-essential amino acids, about 0.1 mM beta-mercaptoethanol and about 4 ng/ml human recombinant basic fibroblast growth factor.
(b) separating the medium from the GLDF cells to obtain conditioned medium.

24. A method of culturing human embryonic stem cells (hESCs), wherein the method comprises culturing hESCs on the conditioned medium of claim 23.

25. The method as claimed in claim 24, wherein the hESCs remain capable of differentiation into ectoderm, mesoderm and endoderm lineages.

26. The method as claimed in claim 24, wherein the human embryonic stem cells are maintained in proliferative and undifferentiated state for about 30-60 passages, preferably about 35 passages.

27. The method as claimed in claim 24, where hESCs are cultured in Petri dish coated with extra cellular matrix selected from a group comprising of Matrigel, fibronectin, poly-L-lysine, laminin, collagen IV, collagen HI and a combination thereof.


SEQUENCE LISTING (40 sequences)

SEQ ID NO: 1 - DNA, 20 bp, Artificial sequence
  A synthetic primer sequence
aacagcgacggaggtctcta

SEQ ID NO: 2 - DNA, 20 bp, Artificial sequence
  A synthetic primer sequence
ttctcttgtcccgcagactt

SEQ ID NO: 3 - DNA, 26 bp, Artificial sequence
  A synthetic primer sequence
cagtccgtcaccctggtgtgcgatgc

SEQ ID NO: 4 - DNA, 27 bp, Artificial Sequence
  A synthetic primer sequence
cagagtctggggtcacctccagatagc

SEQ ID NO: 5 - DNA, 21 bp, Artificial Sequence
  A synthetic primer sequence
cttggggccctgggcctccga

SEQ ID NO: 6 - DNA, 25 bp, Artificial Sequence
  A synthetic primer sequence
gccttcctgcagtggtacacgggcg

SEQ ID NO: 7 - DNA, 20 bp, Artificial Sequence
  A synthetic primer sequence
tgacttctcctgcatgcact

SEQ ID NO: 8 - DNA, 20 bp, Artificial Sequence
  A synthetic primer sequence
agccggcacctgttgtgcaa

SEQ ID NO: 9 - DNA, 20 bp, Artificial Sequence
  A synthetic primer sequence
tccaaaccagaaaacggaag

SEQ ID NO: 10 - DNA, 20 bp, Artificial Sequence
  A synthetic primer sequence
ctgtgcccgtagtgagatga

SEQ ID NO: 11 - DNA, 23 bp, Artificial Sequence
  A synthetic primer sequence
tgtatcgcaggcactcaggtcag

SEQ ID NO: 12 - DNA, 19 bp, Artificial Sequence
  A synthetic primer sequence
aagtctggtcacggggaat

SEQ ID NO: 13 - DNA, 20 bp, Artificial Sequence
  A synthetic primer sequence
gtcctgctaggaggcgcgag

SEQ ID NO: 14 - DNA, 19 bp, Artificial Sequence
  A synthetic primer sequence
gttctccagatgttcttcg

SEQ ID NO: 15 - DNA, 20 bp, Artificial Sequence
  A synthetic primer sequence
tgcctcagaaagagaaccag

SEQ ID NO: 16 - DNA, 20 bp, Artificial Sequence
  A synthetic primer sequence
atggcaggatgaacaaacac

SEQ ID NO: 17 - DNA, 19 bp, Artificial Sequence
  A synthetic primer sequence
tgcaggactcggtggactt

SEQ ID NO: 18 - DNA, 19 bp, Artificial Sequence
  A synthetic primer sequence
tggactcctgctttgcctg

SEQ ID NO: 19 - DNA, 20 bp, Artificial Sequence
  A synthetic primer sequence
acgctgaggaatggttcaag

SEQ ID NO: 20 - DNA, 20 bp, Artificial Sequence
  A synthetic primer sequence
tagacgcctcaatggtttcc

SEQ ID NO: 21 - DNA, 20 bp, Artificial Sequence
  A synthetic primer sequence
gctcgtcgtcgacaacggct

SEQ ID NO: 22 - DNA, 25 bp, Artificial Sequence
  A synthetic primer sequence
caaacatgatctgggtcatcttctc

SEQ ID NO: 23 - DNA, 25 bp, Artificial Sequence
  A synthetic primer sequence
cctcctccatggatctgcttattca

SEQ ID NO: 24 - DNA, 26 bp, Artificial Sequence
  A synthetic primer sequence
caggtcttcacctgtttgtagctgag

SEQ ID NO: 25 - DNA, 18 bp, Artificial Sequence
  A synthetic primer sequence
cccccggcggcaatagca

SEQ ID NO: 26 - DNA, 20 bp, Artificial Sequence
  A synthetic primer sequence
tcggcgccggggagatacat

SEQ ID NO: 27 - DNA, 23 bp, Artificial Sequence
  A synthetic primer sequence
gcgtacgcaaattaaagtccaga

SEQ ID NO: 28 - DNA, 25 bp, Artificial Sequence
  A synthetic primer sequence
cagcatcctaaacagctcgcagaat

SEQ ID NO: 29 - DNA, 24 bp, Artificial Sequence
  A synthetic primer sequence
gcccgcttctcttacagtgtgatt

SEQ ID NO: 30 - DNA, 25 bp, Artificial Sequence
  A synthetic primer sequence
agtacgtgcagacggtggtagttct

SEQ ID NO: 31 - DNA, 20 bp, Artificial Sequence
  A synthetic primer sequence
agctatgcccggacctccat

SEQ ID NO: 32 - DNA, 20 bp, Artificial Sequence
  A synthetic primer sequence
gcctgcagcaggaggatctt

SEQ ID NO: 33 - DNA, 19 bp, Artificial Sequence
  A synthetic primer sequence
gacaagcagcctgtcaagg

SEQ ID NO: 34 - DNA, 19 bp, Artificial Sequence
  A synthetic primer sequence
accatccagcgtgcgttcc

SEQ ID NO: 35 - DNA, 23 bp, Artificial Sequence
  A synthetic primer sequence
gccacatctaatctcatttcaca

SEQ ID NO: 36 - DNA, 20 bp, Artificial Sequence
  A synthetic primer sequence
ctgggtaacagcagatgcaa

SEQ ID NO: 37 - DNA, 22 bp, Artificial Sequence
  A synthetic primer sequence
gggcgcctggtcaccagggctg

SEQ ID NO: 38 - DNA, 21 bp, Artificial Sequence
  A synthetic primer sequence
ggggccatccacagtcttctg

SEQ ID NO: 39 - DNA, 20 bp, Artificial Sequence
  A synthetic primer sequence
cgaccatctgccgctttgag

SEQ ID NO: 40 - DNA, 20 bp, Artificial Sequence
  A synthetic primer sequence
ccccctgtcccccattccta